(19) United States
(12) Patent Application Publication
(10) Pub. No.: US 2006/0257873 A1
(43) Pub. Date: Nov. 16, 2006

(54) Chemical switching of nucleic acid circuit elements

(76) Inventors: Jeremy S. Lee, Saskatchewan (CA); Heinz-Bernhard Kraatz, Saskatchewan (CA); Shawn D. Wettig, Saskatchewan (CA); Ryan John Skinner, Saskatchewan (CA)

Correspondence Address: KNOBBE MARTENS OLSON & BEAR LLP, 2040 MAIN STREET, FOURTEENTH FLOOR, IRVINE, CA, 92614, US

(21) Appl. No.: 10/540,279

(22) PCT Filed: Oct. 15, 2003
(86) PCT No.: PCT/CA03/01589

§ 371 Date: Apr. 14, 2006

Related U.S. Application Data
(60) Provisional application No. 60/435,276, filed on Dec. 23, 2002.

(51) Int. Cl. C12Q 1/68; H01L 21/00; H01L 51/00

(52) U.S. Cl. 435/6; 257/40; 438/1; 977/704

(57) ABSTRACT

Organic circuit elements and organic conductors are disclosed, together with electron acceptors and donors that may be chemically modified to alter the conductivity of the circuit or organic conductor. An organic circuit element includes a plurality of members, each of which includes an oligonucleotide duplex. The plurality of members includes at least one donor member for receiving conduction electrons from an electron donor, at least one acceptor member for communicating with an electron acceptor to provide a region of attraction for the conduction electrons, and at least one regulator member intersecting with at least one of the plurality of members to define at least one electric field regulation junction, for cooperating with an electric field regulator to regulate an electric field at the junction. A method of regulating an electronic signal between first and second locations in a conductive nucleic acid material includes chemically modifying an electron acceptor or an electron donor that is coupled to the conductive nucleic acid material.

### BACKGROUND OF THE INVENTION

1. Field of Invention

The present invention relates to nucleic acids, and more particularly, to organic circuit elements and related methods.

2. Description of Related Art

The field of organic electronics has been given increased attention in an effort to create inexpensive circuit elements which operate on the molecular level to facilitate ever-increasing density requirements of producing smaller circuits. Today's silicon-based microelectronic devices have a minimum size between electrical components of about a tenth of a micron. But in molecular electronics, nanometer-sized components could yield chips exponentially more powerful than anything of a comparable size today or computing devices unimaginably tiny by contemporary standards. Moreover, the search for flexible circuits which are compatible with plastic substrates to produce digitized versions of newspapers, product labels and integrated circuits, for example, has led to the investigation of organic materials as electronic devices.

In this regard, biological materials such as DNA are of interest because of the potential for molecular recognition and the ability to synthesize them using biological machinery. Moreover, due to its importance in living organisms, DNA has been subjected to a wide range of structural, kinetic, and thermodynamic probes (Gelbart et al., 2000). However, recently, measurements of electrical transport through individual short DNA molecules indicate wide-band gaps semiconductor behavior (Porath et al., 2000), while other measurements of DNA hairpins have indicated that DNA is only somewhat more effective than proteins as a conductor of electrons (Lewis et al., 1997; Taubes, 1997). U.S. Pat. Nos. 5,591,578; 5,705,348; 5,770,369; 5,780,234 and 5,824,473 issued to Meade et al. on, respectively, 7 Jan. 1997, 6 Jan. 1998, 23 Jun. 1998, 14 Jul. 1998 and 20 Oct. 1998 (and incorporated herein by reference) disclose nucleic acids that are covalently modified with electron transfer moieties along the nucleic acid backbone. Meade et al. suggest that such modifications are necessary for nucleic acids to efficiently mediate electron transfer.

A new form of conductive nucleic acid has recently been found which is described in International Patent Publication WO 99/31115, Aich et al., 1999, and Rakitin et al., 2000, all of which are incorporated herein by reference. M-DNA is a novel conformation of duplex DNA in which the imino protons of each base pair are replaced by a metal ion (such as Zn²⁺, Ni²⁺ or Co²⁺). It has been shown by two independent methods (Aich et al., 1999, and Rakitin et al., 2000) that M-DNA conducts electrons in contrast to normal duplex DNA, which is reportedly a semiconductor at best. Direct measurements of the conductivity of M-DNA were performed by stretching phage λ-DNA between two electrodes separated by 3 to 10 microns (Rakitin et al., 2000). Indirect measurements of the conductivity were estimated from fluorescent lifetime measurements of duplexes with a donor fluorophore at one end and an acceptor fluorophore at the other (Rakitin et al., 2000, Aich et al., 1999). Upon conversion to M-DNA, the fluorescein of the donor was quenched and the lifetime was so short as to be only consistent with an electron transfer mechanism. The transfer of electrons from excited fluorophores indicates that M-DNA may for example be used in some embodiments as a molecular wire.

### SUMMARY OF THE INVENTION

In accordance with one aspect of the invention, there is provided an organic circuit element. The circuit element includes a plurality of members, each of which includes an oligonucleotide duplex. The plurality of members includes at least one donor member for receiving conduction electrons from an electron donor, at least one acceptor member for communicating with an electron acceptor to provide a region of attraction for the conduction electrons, and at least one regulator member intersecting with at least one of the plurality of members to define at least one electric field regulation junction, for cooperating with an electric field regulator to regulate an electric field at the junction.

At least some of the plurality of members may include a conductive metal-containing oligonucleotide duplex. For example, each of the members may include such a conductive metal-containing oligonucleotide duplex. Alternatively, the at least one donor member and the at least one acceptor member may include such a conductive metal-containing oligonucleotide duplex.

The organic circuit element may further include the electron donor in electrical communication with the donor member. Similarly, the organic circuit element may include the electron acceptor in electrical communication with the acceptor member. Alternatively, or in addition, the organic circuit element may include the electric field regulator in electrical communication with the regulator member.

The donor member, the acceptor member and the regulator member may intersect to define the electric field regulation junction.

Alternatively, the regulator member may intersect with one of the donor member and the acceptor member to define the electric field regulation junction.

Alternatively, the plurality of members may include a common member, and the donor member, the acceptor member and the regulator member may intersect the common member at first, second and third locations respectively, the third location defining the electric field regulation junction.

The at least one regulator member may include a plurality of regulator members, the plurality of regulator members intersecting other respective members of the plurality of members to define the at least one electric field regulation junction.

The conductive metal-containing oligonucleotide duplex may include a first nucleic acid strand and a second nucleic acid strand, the first and second nucleic acid strands including respective pluralities of nitrogen-containing aromatic bases covalently linked by a backbone. The nitrogen-containing aromatic bases of the first nucleic acid strand may be joined by hydrogen bonding to the nitrogen-containing aromatic bases of the second nucleic acid strand. The nitrogen-containing aromatic bases on the first and the second nucleic acid strands may form hydrogen-bonded base pairs in stacked arrangement along a length of the conductive metal-containing oligonucleotide duplex. The hydrogen-bonded base pairs may include an interchelated metal cation coordinated to a nitrogen atom in one of the nitrogen-containing aromatic bases.

The interchelated metal cation may include an interchelated divalent metal cation.

The divalent metal cation may be selected from the group consisting of zinc, cobalt and nickel.

Alternatively, the metal cation may be selected from the group consisting of the cations of Li, Be, Na, Mg, Al, K, Ca, Sc, Ti, V, Cr, Mn, Fe, Co, Ni, Cu, Zn, Ga, Ge, As, Rb, Sr, Y, Zr, Nb, Mo, Tc, Ru, Rh, Pd, Ag, Cd, In, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U, Np and Pu.

The first and the second nucleic acid strands may include deoxyribonucleic acid and the nitrogen-containing aromatic bases may be selected from the group consisting of adenine, thymine, guanine and cytosine.

The divalent metal cations may be substituted for imine protons of the nitrogen-containing aromatic bases, and the nitrogen-containing aromatic bases may be selected from the group consisting of thymine and guanine.

If desired, at least one of the nitrogen-containing aromatic bases may include thymine, having an N3 nitrogen atom, and the divalent metal cation may be coordinated by the N3 nitrogen atom.

Alternatively, if desired, at least one of the nitrogen-containing aromatic bases may include guanine, having an N1 nitrogen atom, and the divalent metal cation may be coordinated by the N1 nitrogen atom.

The electron donor may include an electrode operable to donate an electron to the donor member.

Alternatively, or in addition, the electron donor may include an electron donor molecule capable of donating an electron to the donor member. The electron donor molecule may include a fluorescent molecule, such as fluorescein, for example.

The electron acceptor may include an electrode operable to accept an electron from the acceptor member.

Alternatively, or in addition, the electron acceptor may include an electron acceptor molecule capable of accepting an electron from the acceptor member. The electron acceptor molecule may include a fluorescent molecule, such as rhodamine, for example.

The electric field regulator may include a regulator chromophore. The regulator chromophore may absorb radiation within a range of wavelengths.

The electric field regulator may include a fluorescent molecule, such as fluorescein or rhodamine, for example.

The electron acceptor may include a chromophore operable to emit radiation within a range of wavelengths in response to accepting an electron from the acceptor member.

In some embodiments, the electron donor or acceptor moieties may be chemically altered to change the electrical properties of the nucleic acid circuit element. The donors or acceptors may for example be reversibly reduced or oxidized under conditions that preserve the potential for conductivity of the M-DNA.

The electric field regulator may include an electrode, which may be operable to perform at least one of accepting an electron from the acceptor member and donating an electron to the donor member.

The electric field regulator may include a plurality of states, each state of the plurality of states being selectable to produce a respective electrostatic potential at the electric field regulation junction. The states may be selectable in response to an applied external potential, or by irradiating the electric field regulator, for example.

In accordance with another aspect of the invention, there is provided a system including an organic circuit element as described above, and further including a conductive medium for supplying conduction electrons to the electron donor and for receiving conduction electrons from the electron acceptor.

The conductive medium may be operable to donate electrons to the electron donor, and may be operable to accept electrons from the electron acceptor to provide a closed circuitway for electrons to flow from the electron donor, through the donor member, through the electric field regulation junction, through the acceptor member, through the electron acceptor, and back to the electron donor.

The conductive medium may include an aqueous solution. Or, the conductive medium may include a conductive wire.

In accordance with another aspect of the invention, there is provided a method of making an organic circuit element. The method includes annealing and treating a plurality of oligonucleotides to form a plurality of members, each member of the plurality of members including a pair of the oligonucleotides aligned to form a duplex portion. The plurality of members includes at least one donor member for receiving conduction electrons from an electron donor, at least one acceptor member for communicating with an electron acceptor to provide a region of attraction for the conduction electrons, and at least one regulator member intersecting with at least one of the plurality of members to define at least one electric field regulation junction, for cooperating with an electric field regulator to regulate an electric field at the junction.

The method may further include placing the electron donor in electrical communication with the donor member. Similarly, the method may include placing the electron acceptor in electrical communication with the acceptor member. Additionally, or alternatively, the method may include placing the electric field regulator in electrical communication with the regulator member.

Annealing and treating may include annealing and treating the plurality of oligonucleotides to form the plurality of members in a configuration in which the donor member, the acceptor member and the regulator member intersect to define the electric field regulation junction.

Alternatively, annealing and treating may include annealing and treating the plurality of oligonucleotides to form the plurality of members in a configuration in which the regulator member intersects with one of the donor member and the acceptor member to define the electric field regulation junction.

Alternatively, the plurality of members may include a common member, and wherein annealing and treating include annealing and treating the plurality of oligonucleotides to form the plurality of members in a configuration in which the donor member, the acceptor member and the regulator member intersect the common member at first, second and third locations respectively, the third location defining the electric field regulation junction.

The plurality of members may include a plurality of regulator members, in which case annealing and treating may include annealing and treating the plurality of oligonucleotides to form the members in a configuration in which the plurality of regulator members intersect the plurality of members to define the at least one electric field regulation junction.

Annealing may include annealing the plurality of oligonucleotides in conditions effective to form the duplex portion, and treating may include treating the plurality of oligonucleotides in conditions effective to form the at least one electric field regulation junction.

The oligonucleotides may include a plurality of nitrogen-containing aromatic bases covalently linked by a backbone.

The oligonucleotides may include a deoxyribonucleic acid including nitrogen-containing aromatic bases selected from the group consisting of adenine, thymine, guanine, cytosine, and uracil.

The duplex portion may include a conductive metal-containing oligonucleotide duplex portion, the conductive metal-containing oligonucleotide duplex portion including a first strand and a second strand of the oligonucleotides, the nitrogen-containing aromatic bases of the first strand joined by hydrogen bonding to the nitrogen-containing aromatic bases of the second strand, the nitrogen-containing aromatic bases on the first and second strands forming hydrogen-bonded base pairs in stacked arrangement along a length of the conductive metal-containing oligonucleotide duplex portion, the hydrogen-bonded base pairs including an interchelated metal cation coordinated to a nitrogen atom in one of the nitrogen-containing aromatic bases.

The interchelated metal cation may include an interchelated divalent metal cation.

Annealing may include subjecting the plurality of oligonucleotides to a basic solution under conditions effective to form the conductive metal-containing oligonucleotide duplex portion.

The conditions effective to form the conductive metal-containing oligonucleotide duplex portion may include conditions effective to substitute the divalent metal cations for an imine proton of a nitrogen containing aromatic base in the conductive metal-containing oligonucleotide duplex portion.

The basic solution may have a pH of at least 7, and may have a nucleic acid to metal ion ratio of about 1:1.5 to about 1:2.0, for example.

The divalent metal cation may be selected from the group consisting of zinc, cobalt and nickel.

Alternatively, the metal cation may be selected from the group consisting of the cations of Li, Be, Na, Mg, Al, K, Ca, Sc, Ti, V, Cr, Mn, Fe, Co, Ni, Cu, Zn, Ga, Ge, As, Rb, Sr, Y, Zr, Nb, Mo, Tc, Ru, Rh, Pd, Ag, Cd, In, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, TI, Pb, Bi, Po, Fr, Ra, Ac, Th, Pa, U, Np and Pu. For example, in some embodiments, varying amounts of metal cations may be incorporated into a duplex, such as Zn²⁺, Ni²⁺, Co²⁺, Cd²⁺, Hg²⁺, Pt²⁺ and Ag¹⁺, where metal ions such as Cd²⁺, Hg²⁺, Pt²⁺ and Ag¹⁺ may constitute only a portion of the metal ions in the duplex, in effect ‘doping’ the duplex.

The divalent metal cations may be substituted for imine protons of the nitrogen-containing aromatic bases, and the nitrogen-containing aromatic bases may be selected from the group consisting of thymine and guanine.

If desired, at least one of the nitrogen-containing aromatic bases may include thymine, having an N3 nitrogen atom, and the divalent metal cation may be coordinated by the N3 nitrogen atom.

Similarly, at least one of the nitrogen-containing aromatic bases may include guanine, having an N1 nitrogen atom, and the divalent metal cation may be coordinated by the N1 nitrogen atom.

The electron donor may include an electron donor molecule capable of donating an electron to the donor member. Similarly, the electron acceptor may include an electron acceptor molecule capable of accepting an electron from the acceptor member.

The electron donor molecule may include a fluorescent molecule, such as fluorescein, for example.

Similarly, the electron acceptor molecule may include a fluorescent molecule, such as rhodamine, for example.

Alternatively, the electron donor may include an electrode operable to donate an electron to the donor member.

Similarly, the electron acceptor may include an electrode operable to accept an electron from the acceptor member.

The electric field regulator may include a fluorescent molecule, such as fluorescein or rhodamine, for example.

The electric field regulator may include a regulator chromophore. If so, the regulator chromophore may absorb radiation within a range of wavelengths.

The electron acceptor may include a chromophore operable to emit radiation within a range of wavelengths in response to accepting an electron from the acceptor member.

Treating may include subjecting the plurality of oligonucleotides to a basic solution under conditions effective to form the electric field regulation junction.

The electric field regulator may include an electrode, which may be operable to perform at least one of accepting an electron from the acceptor member and donating an electron to the donor member.

The electric field regulator may include a plurality of states, each state of the plurality of states being selectable to produce a respective electrostatic potential at the electric field regulation junction.

In accordance with another aspect of the invention, there is provided a method of regulating an electronic signal between first and second locations in a conductive nucleic acid material. The method includes varying an electrostatic potential at a third location in the nucleic acid material interposed between the first and second locations.

Varying may include selecting one of a plurality of states of an electric field regulator in communication with the third location, each of the states corresponding to a respective electrostatic potential at the third location.

Selecting may include irradiating the electric field regulator. For example, if the electric field regulator includes a chromophore, or is selected from the group consisting of fluorescent molecules and chromophores, selecting may include irradiating the electric field regulator.

Irradiating may include irradiating the chromophore to cause a negative electrostatic potential to be applied to the third location.

Alternatively, selecting may include applying an external potential to the electric field regulator. For example, if the electric field regulator includes an electrode, and selecting may include applying an external potential to the electrode.

Applying may include depositing at least one electron onto the electrode to apply a negative electrostatic potential to the third location.

Conversely, applying may include removing at least one electron from the electrode to apply a positive electrostatic potential to the third location.

The method may further include producing the electronic signal. This may include causing electrons to flow from the first location to the second location, and may further include supplying electrons to the first location and receiving electrons from the second location, for example.

The first location may include a location in a conductive nucleic acid electron donor member, the second location may include a location in a conductive nucleic acid electron acceptor member, and the third location may include at least one electric field regulation junction in electrical communication with the donor member and the acceptor member. If so, then varying may include varying the electrostatic potential at the at least one electric field regulation junction.

The at least one electric field regulation junction may be in electrical communication with a conductive nucleic acid electric field regulator member. In such a case, varying may include selecting one of a plurality of states of an electric field regulator in electrical communication with the regulator member, each of the states corresponding to a respective electrostatic potential at the at least one electric field regulation junction.

As noted above, selecting may include irradiating the electric field regulator, for example, where the regulator is selected from the group consisting of fluorescent molecules and chromophores, or is a chromophore. In the latter case, irradiating may include irradiating the chromophore to cause a negative electrostatic potential to be applied to the electric field regulation junction, the negative electrostatic potential decreasing the ability of an electron to travel from the donor member to the acceptor member.

Alternatively, selecting may include applying an external potential to the electric field regulator, for example, where the regulator includes an electrode. In the latter case, applying may include depositing at least one electron onto the electrode to apply a negative electrostatic potential to the electric field regulation junction, the negative electrostatic potential decreasing the ability of an electron to travel from the donor member to the acceptor member. Conversely, applying may include removing at least one electron from the electrode to apply a positive electrostatic potential to the electric field regulation junction, the positive electrostatic potential increasing the ability of an electron to travel from the donor member to the acceptor member.

The method may further include placing the electron donor member, the electron acceptor member, and the regulator member in electrical communication with an electron donor, an electron acceptor, and the electric field regulator, respectively.

The method may further include producing the electronic signal. Producing may include causing electrons to flow from an electron donor in communication with the electron donor member, to an electron acceptor in communication with the electron acceptor member. The method may further include supplying electrons to the electron donor and receiving electrons from the electron acceptor.

The at least one electric field regulation junction may include at least two electric field regulation junctions in electrical communication with at least two respective electric field regulators. If so, then wherein varying may include selecting one of a plurality of states of at least one of the at least two electric field regulators, each of the states corresponding to a respective electrostatic potential at the electric field regulation junction corresponding to the at least one of the at least two electric field regulators.

The conductive nucleic acid material may include a plurality of members, each of which may include a conductive metal-containing oligonucleotide duplex. The plurality of members may include at least one donor member for receiving conduction electrons from an electron donor, at least one acceptor member for communicating with an electron acceptor to provide a region of attraction for the conduction electrons, and at least one regulator member intersecting with at least one of the plurality of members to define at least one electric field regulation junction, for cooperating with an electric field regulator to regulate an electric field at the junction. In such a case, varying may include selecting one of a plurality of states of the electric field regulator, each of the states corresponding to a respective electrostatic potential at the electric field regulation junction.

The conductive nucleic acid material may include a conductive metal-containing nucleic acid duplex. The duplex may include a regulator member in electrical communication with an electric field regulator, a donor member in electrical communication with an electron donor, and an acceptor member in electrical communication with an electron acceptor. In such a case, varying may include changing the state of the electric field regulator to vary an electrostatic potential at an electric field regulation junction joining the regulator member, the donor member, and the acceptor member, to regulate the signal.

The conductive metal-containing nucleic acid duplex may include a nucleic acid duplex including a first nucleic acid strand and a second nucleic acid strand. The first and the second nucleic acid strands may include respective pluralities of nitrogen-containing aromatic bases covalently linked by a backbone. The nitrogen-containing aromatic bases of the first nucleic acid strand may be joined by hydrogen bonding to the nitrogen-containing aromatic bases of the second nucleic acid strand. The nitrogen-containing aromatic bases on the first and the second nucleic acid strands may form hydrogen-bonded base pairs in stacked arrangement along a length of the nucleic acid duplex.

The method may further include producing the conductive metal-containing nucleic acid duplex. Producing may include subjecting the nucleic acid duplex to a basic solution in the presence of a metal cation under conditions effective to form the conductive metal-containing nucleic acid duplex, wherein the hydrogen-bonded base pairs of the conductive metal-containing nucleic acid duplex include an interchelated metal cation coordinated to a nitrogen atom in one of the nitrogen-containing aromatic bases.

More particularly, producing may include subjecting the nucleic acid duplex to a basic solution in the presence of a divalent metal cation under conditions effective to form the conductive metal-containing nucleic acid duplex, wherein the hydrogen-bonded base pairs of the conductive metal-containing nucleic acid duplex include an interchelated divalent metal cation coordinated to a nitrogen atom in one of the nitrogen-containing aromatic bases.

The nucleic acid duplex may include a deoxyribonucleic acid duplex including nitrogen-containing aromatic bases selected from the group consisting of adenine, thymine, guanine and cytosine.

The conditions effective to form the conductive metal-containing nucleic acid duplex may be effective to substitute the divalent metal cations for an imine proton of a nitrogen containing aromatic base in the nucleic acid duplex.

The divalent metal cation may be selected from the group consisting of zinc, cobalt and nickel. Alternatively, the metal cation may be selected from the group consisting of the cations of Li, Be, Na, Mg, Al, K, Ca, Sc, Ti, V, Cr, Mn, Fe, Co, Ni, Cu, Zn, Ga, Ge, As, Rb, Sr, Y, Zr, Nb, Mo, Tc, Ru, Rh, Pd, Ag, Cd, In, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, TI, Pb, Bi, Po, Fr, Ra, Ac, Th, Pa, U, Np and Pu.

The basic solution may have a pH of at least 7, and may have a nucleic acid to metal ion ratio of about 1:1.5 to about 1:2.0, for example.

The electron donor may include an electron donor molecule capable of donating an electron to the donor member. The electron donor molecule may include a fluorescent molecule, such as fluorescein, for example.

Similarly, the electron acceptor may include an electron acceptor molecule capable of accepting an electron from the acceptor member. The electron acceptor molecule may include a fluorescent molecule, such as rhodamine, for example.

Alternatively, or in addition, the electron donor may include an electrode operable to donate an electron to the donor member. Similarly, the electron acceptor may include an electrode operable to accept an electron from the acceptor member.

The electric field regulator may include a regulator chromophore, or a fluorescein, or a rhodamine, for example. The regulator chromophore may absorb radiation within a range of wavelengths.

The electron acceptor may include a chromophore operable to emit radiation within a range of wavelengths in response to accepting an electron from the acceptor member. The radiation may irradiate a second chromophore in series.

Any or all of the regulator member, the donor member and the acceptor member may include a conductive metal-containing nucleic acid duplex portion.

The method may further include supplying conduction electrons from a conductive medium to the conductive metal-containing nucleic acid duplex, and receiving conduction electrons from the duplex at the conductive medium. Supplying may include donating electrons from the conductive medium to the electron donor, and receiving may include accepting electrons from the electron acceptor at the conductive medium, to provide a closed circuitway for electrons to flow from the electron donor, through the donor member, through the electric field regulation junction, through the acceptor member, through the electron acceptor, and through the conductive medium to the electron donor. The conductive medium may include an aqueous solution, or may include a conductive wire, for example.

Changing the state of the electric field regulator may include irradiating the regulator chromophore to cause a negative electrostatic potential to be produced and applied to the electric field regulation junction, the negative electrostatic potential decreasing the ability of an electron to travel from the donor member to the acceptor member.

The electric field regulator may include an electrode, which may, be operable to perform at least one of accepting an electron from the acceptor member and donating an electron to the donor member.

Changing the state of the electric field regulator may include depositing an electron onto the electrode to produce a negative electrostatic potential applied to the electric field regulation junction, the negative electrostatic potential decreasing the ability of an electron to travel from the donor member to the acceptor member.

Conversely, changing the state of the electric field regulator may include removing an electron from the electrode to produce a positive electrostatic potential applied to the electric field regulation junction, the positive electrostatic potential increasing the ability of an electron to travel from the donor member to the acceptor member.

The electric field regulator may include a plurality of states, each state of the plurality of states being selectable in response to an applied external potential to produce a respective electrostatic potential at the electric field regulation junction.

In accordance with another aspect of the invention, there is provided an apparatus for regulating an electronic signal between first and second locations in a conductive nucleic acid material. The apparatus includes the conductive nucleic acid material having the first and second locations, and further includes means for varying an electrostatic potential at a third location in the nucleic acid material interposed between the first and second locations.

The means for varying may include means for selecting one of a plurality of states of an electric field regulator in communication with the third location, each of the states corresponding to a respective electrostatic potential at the third location.

The means for selecting may include means for irradiating the electric field regulator.

Alternatively, the means for selecting may include means for applying an external potential to the electric field regulator.

The electric field regulator may include an electrode, in which case the means for applying may include means for depositing at least one electron onto the electrode to apply a negative electrostatic potential to the third location.

Alternatively, or in addition, the means for applying may include means for removing at least one electron from the electrode to apply a positive electrostatic potential to the third location.

The apparatus may further include means for producing the electronic signal.

The first location may include a location in a conductive nucleic acid electron donor member, the second location may include a location in a conductive nucleic acid electron acceptor member, and the third location may include at least one electric field regulation junction in electrical communication with the donor member and the acceptor member. In such a case, the means for varying may include means for varying the electrostatic potential at the at least one electric field regulation junction.

The least one electric field regulation junction may be in electrical communication with a conductive nucleic acid electric field regulator member. If so, the means for varying may include means for selecting one of a plurality of states of an electric field regulator in electrical communication with the regulator member, each of the states corresponding to a respective electrostatic potential at the at least one electric field regulation junction.

The means for selecting may include means for irradiating the electric field regulator.

Alternatively, the means for selecting may include means for applying an external potential to the electric field regulator. For example, the electric field regulator may include an electrode, and the means for applying may include means for depositing at least one electron onto the electrode to apply a negative electrostatic potential to the electric field regulation junction, the negative electrostatic potential decreasing the ability of an electron to travel from the donor member to the acceptor member. Alternatively, or in addition, the means for applying may include means for removing at least one electron from the electrode to apply a positive electrostatic potential to the electric field regulation junction, the positive electrostatic potential increasing the ability of an electron to travel from the donor member to the acceptor member.

In accordance with another aspect of the invention, there is provided an apparatus for regulating an electronic signal between first and second locations in a conductive nucleic acid material. The apparatus includes an electric field regulator operable to vary an electrostatic potential at a third location in the nucleic acid material interposed between the first and second locations.

The electric field regulator may have a plurality of selectable states, each of the states corresponding to a respective electrostatic potential at the third location.

The electric field regulator may include an electrode. Alternatively, the electric field regulator may include a chromophore, or may include a fluorescent molecule such as fluorescein or rhodamine for example, or may be selected from the group consisting of fluorescent molecules and chromophores, for example.

The first location may include a location in a conductive nucleic acid electron donor member, the second location may include a location in a conductive nucleic acid electron acceptor member, and the third location may include at least one electric field regulation junction in electrical communication with the donor member, the acceptor member, and the electric field regulator.

The apparatus may further include a regulator member joining the electric field regulator to the electric field regulation junction.

In accordance with another aspect of the invention, there is provided a method of regulating an electronic signal in a conductive nucleic acid material. The method includes varying a degree of electric field regulation at an electric field regulation junction at which a regulator member intersects at least one of a plurality of members. Each of the regulator member and the plurality of members includes an oligonucleotide duplex, and at least some of the regulator member and the plurality of members includes a conductive metal-containing oligonucleotide duplex. The plurality of members includes at least one donor member for receiving conduction electrons from an electron donor, and at least one acceptor member for communicating with an electron acceptor to provide a region of attraction for the conduction electrons.

Varying may include varying an electrostatic potential at the electric field regulation junction.

Varying may include selecting one of a plurality of states of an electric field regulator in communication with the electric field regulation junction via the regulator member.

Selecting may include irradiating the electric field regulator, or may include applying an external potential to the electric field regulator, for example.

In accordance with another aspect of the invention, there is provided a method of storing data. The method includes selecting one of at least two states of an electric field regulator of a nucleic acid circuit element, each of the at least two states corresponding to a respective degree of electric field regulation at an electric field regulation junction in the circuit element, each degree of electric field regulation corresponding to a respective data value.

Selecting may include irradiating the electric field regulator, or may include applying an external potential to the electric field regulator, for example.

The nucleic acid circuit element may include a plurality of members, at least some of which may include a conductive metal-containing oligonucleotide duplex. The plurality of members may include at least one donor member for receiving conduction electrons from an electron donor, at least one acceptor member for communicating with an electron acceptor to provide a region of attraction for the conduction electrons, and at least one regulator member intersecting with at least one of the plurality of members to define the electric field regulation junction, the regulator member being in communication with the electric field regulator. In such a case, selecting may include causing the electric field regulation junction to apply the degree of electric field regulation to the electric field regulation junction, to represent the data value.

In accordance with another aspect of the invention, there is provided an organic data storage medium. The medium includes an electric field regulator having at least two selectable states, each of the states corresponding to a respective degree of electric field regulation at an electric field regulation junction of a nucleic acid circuit element, each degree of electric field regulation corresponding to a respective data value.

The organic data storage medium may further include the nucleic acid circuit element, which in turn may include a plurality of members, at least some of which may include a conductive metal-containing oligonucleotide duplex. The plurality of members may include at least one donor member for receiving conduction electrons from an electron donor, at least one acceptor member for communicating with an electron acceptor to provide a region of attraction for the conduction electrons, and at least one regulator member intersecting with at least one of the plurality of members to define the electric field regulation junction, for cooperating with the electric field regulator to apply the degree of electric field regulation to the junction, to represent the data value.

The at least two states may be selectable by irradiating the electric field regulator, or by applying an external potential to the electric field regulator, for example.

Each of the at least two states may correspond to a respective electrostatic potential at the electric field regulation junction.

In accordance with another aspect of the invention, there is provided an apparatus for storing data. The apparatus includes a conductive nucleic acid circuit element comprising an electric field regulation junction, and further includes means for varying a degree of electric field regulation at the electric field regulation junction in the circuit element, each degree of electric field regulation corresponding to a respective data value.

The means for varying may include means for varying an electrostatic potential at the electric field regulation junction.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 is a graphical representation of an organic circuit element according to a first embodiment of the invention.

FIG. 2 is a pictorial representation of a modeled structure of M-DNA as part of the organic circuit element depicted in FIG. 1.

FIG. 3 is a pictorial depiction of a base pair scheme for M-DNA shown in FIG. 2 as part of the organic circuit element of FIG. 1, according to the first embodiment of the invention.

FIG. 4 is a pictorial depiction of a base pairing scheme for M-DNA shown in FIG. 2 as part of the organic circuit element shown in FIG. 1, according to a second embodiment of the invention.

FIG. 5 is a graphical representation of current voltage characteristics measured on M-DNA shown in FIG. 2 and B-DNA at room temperature. The lower inset shows a schematic diagram of an experimental layout used to produce I-V characteristics.

FIG. 6 is a graphical representation of an organic circuit element according to a third embodiment of the invention.

FIG. 7 is a graphical representation of an organic circuit element according to a fourth embodiment of the invention.

FIG. 8 is a graphical representation of an organic circuit element according to a fifth embodiment of the invention.

FIG. 9 is a graphical representation of an organic circuit element according to a sixth embodiment of the invention.

FIG. 10 is a graphical representation of an organic circuit element according to a seventh embodiment of the invention.

FIG. 11: a) Structures of 9,10-anthraquinone-2-carboxylic acid and 9,10-dihydroanthraquinone-2-carboxylic acid, and b) schematic of the Y-branched junctions.

FIG. 12: Absorbance spectra of 30 μM AQ-NHS is 20 mM Tris-HCl, pH 8.5 buffer; 0 mM NaBH₄ (solid), 2.5 mM NaBH₄ (dashed), +O₂ (dotted). The normalized emission spectrum of fluorescein (dash-dot) is included for reference.

FIG. 13: Electrophorgram demonstrating the effect of anthraquinone reduction on the FI-30-Aq duplex. Lane 1, DNA Molecular Weight Marker VIII; lane 2, empty; lanes 3 and 6, 2.5 mM NaBH₄; lane 4, 25 mM NaBH₄; lanes 5 and 7, 0 mM NaBH₄. For lanes 3-5, reduction carried out prior to hybridization; for lanes 6-7, reduction carried out after hybridization.

FIG. 14: Normalized fluorescence for the Fluorescein/Anthraquinone labeled 30-mer as a function of NaBH4 used to reduce the AQ-labeled single strand. The reduction procedure was carried out prior to hybridization. For all measurements [DNA]=0.5 μg mL⁻¹; [Zn²⁺]=0.2 mM; pH 8.49 in 20 mM Tris-HCl buffer.

### DETAILED DESCRIPTION

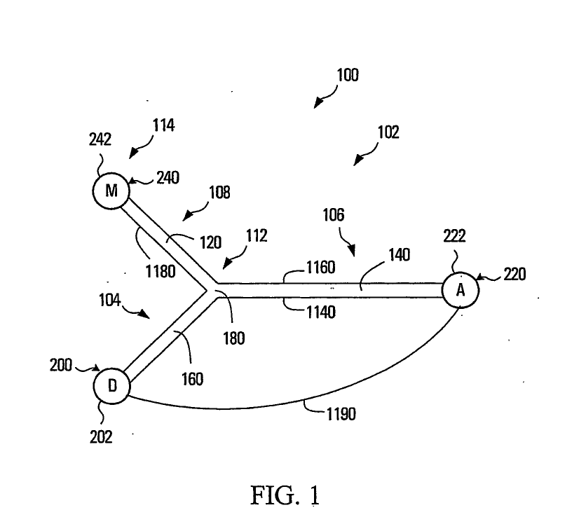

Referring to FIG. 1, an organic circuit element according to a first embodiment of the invention is shown generally at 100. In this embodiment, the organic circuit element 100 includes a plurality 102 of members, each of which includes an oligonucleotide duplex. More particularly, in this embodiment the plurality 102 of members includes at least one donor member 104 for receiving conduction electrons from an electron donor 200, and at least one acceptor member 106 for communicating with an electron acceptor 220 to provide a region of attraction for the conduction electrons. In this embodiment, the plurality 102 of members further includes at least one regulator member 108 intersecting with at least one of the plurality 102 of members to define at least one electric field regulation junction 112, for cooperating with an electric field regulator 114 to regulate an electric field at the electric field regulation junction 112.

In this embodiment, at least some of the plurality of members include a conductive metal-containing oligonucleotide duplex. More particularly, in this embodiment, each of the plurality of members includes a conductive metal-containing oligonucleotide duplex.

In the present embodiment, the plurality 102 of members includes a plurality of arms. More particularly, in this embodiment the donor member 104 includes a donor arm 160 electrically coupled to the electron donor 200 (“D”) to provide a source of conduction electrons. The acceptor member 106 of the present embodiment includes an acceptor arm 140 electrically coupled to the electron acceptor 220 (“A”) to provide a region of attraction for the conduction electrons. In this embodiment, the regulator member 108 includes a modulator arm 120 electrically coupled to the electric field regulator 114, which in this embodiment includes an electron flow modulator 240 (“M”) to regulate the flow of the conduction electrons from the electron donor, through the electric field regulation junction 112, to the electron acceptor 220.

In this embodiment, the donor member 104, the acceptor member 106 and the regulator member 108 intersect to define the electric field regulation junction 112. Thus, in the present embodiment the electric field regulation junction 112 includes a conductive junction 180, which forms a three-arm junction connecting the arms 120, 140 and 160, which extend from the conductive junction. However, the conductive junction may include more than three members in alternative embodiments.

In this embodiment, the organic circuit element 100 includes the electric field regulator 114 in electrical communication with the regulator member 108, the electron donor 200 in electrical communication with the donor member 104, and the electron acceptor 220 in electrical communication with the acceptor member 106.

In the present embodiment, the electric field regulator 114 includes a plurality of selectable states, each of the states corresponding to a respective electrostatic potential at the at least one electric field regulation junction 112. More particularly, in the present embodiment, the electric field regulator 114, which in this embodiment includes the electron flow modulator 240, has various states, each state of the plurality of states being selectable in response to an applied external potential to produce a respective electrostatic potential at the electric field regulation junction 112. Alternatively, the states of the electron flow modulator may be selectable or changeable in any other suitable way, such as by irradiating the electron flow modulator for example, as discussed in greater detail below.

In various exemplary embodiments, the state of the electron flow modulator 240 may for example be any macroscopic- or microscopic variable effective in determining the quantum-mechanical wave function of the electron flow modulator. For example, the state of the electron flow modulator 240 may represent the number of electrons added to or removed from the electron flow modulator, or the magnitude and/or direction of an external potential applied to the electron flow modulator. Moreover, the state of the electron flow modulator 240 may represent the orbital level of a valence electron on the electron flow modulator, or further properties of the orbital, such as a degeneracy level. Alternatively or in addition, the state of the electron flow modulator 240 may include a total spin of the electrons on the electron flow modulator or any other parameter sets indicating the quantum mechanical wave function identifying the state of the electron flow modulator.

The state of the electron flow modulator 240 may be selectable or changeable to vary an electrostatic potential at the conductive junction 180, joining the modulator arm 120, the donor arm 160, and the acceptor arm 140, to regulate electron flow or conductivity from the electron donor 200 to the electron acceptor 220. The state of the electron flow modulator 240 may be changeable, for example, by applying an external potential to the electron flow modulator or depositing or removing electrons to or from its outer valence orbitals. Electron flow may represent an electronic signal, such as electron transport as in a DC signal, or a modulated voltage or current signal, or any other signal modulated to carry information. Thus, when the state of the electron flow modulator 240 is changed to vary the electrostatic potential at the conductive junction 180, the electron flow or conductivity from the electron donor 200 to the electron acceptor 220 through the conductive junction 180 may be modulated to thereby regulate a signal passed from the electron donor arm to the electron acceptor arm.

In this embodiment, the organic circuit element 100 includes a conductive nucleic acid material. More particularly, in the present embodiment, each of the donor member 104, the regulator member 108 and the acceptor member 106 includes a conductive metal-containing nucleic acid duplex portion. More particularly still, in this embodiment the donor arm 160, the modulator arm 120 and the acceptor arm 140 each includes a conductive metal-containing oligonucleotide duplex which is able to conduct electrons.

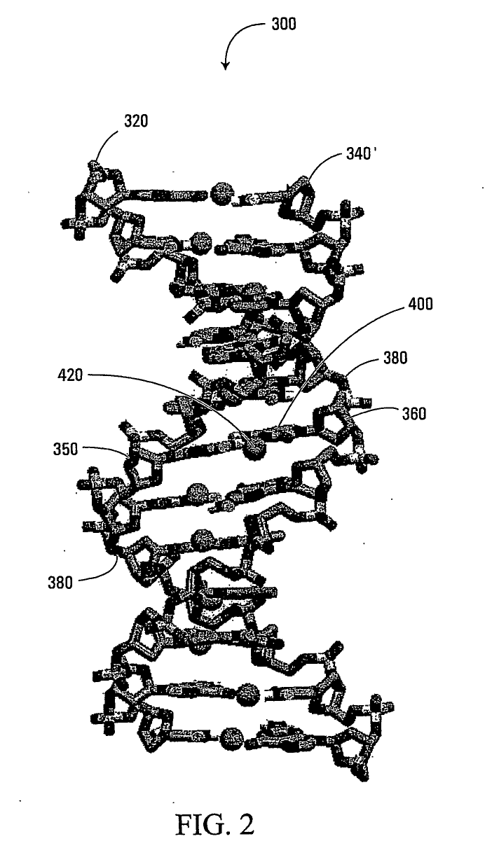

An example of a conductive metal-containing oligonucleotide duplex (“M-DNA”) is shown at 300 in FIG. 2. In this embodiment, the M-DNA 300 includes a first nucleic acid strand 320 and a second nucleic acid strand 340. The first and second nucleic acid strands 320 and 340 include respective pluralities of nitrogen-containing aromatic bases 350 and 360, covalently linked by a backbone 380. The nitrogen-containing aromatic bases 350 of the first nucleic acid strand 320 are joined by hydrogen bonding to the nitrogen-containing aromatic bases 360 of the second nucleic acid strand 340. The nitrogen-containing aromatic bases 350 and 360 on the first and the second nucleic acid strands 320 and 340, respectively, form hydrogen bonded base pairs 400 in stacked arrangement along a length of the conductive metal-containing oligonucleotide duplex 300. The hydrogen-bonded base pairs 400 include an interchelated metal cation 420 coordinated to a nitrogen atom in one of the nitrogen-containing aromatic bases 350 or 360. More particularly, in this embodiment the interchelated metal cation includes an interchelated divalent metal cation. In the present embodiment, the first and second nucleic acid strands 320 and 340 respectively include deoxyribonucleic acid and the nitrogen-containing aromatic bases 350 and 360 are selected from the group consisting of adenine, thymine, guanine and cytosine.

Alternatively, other backbone structures 380 may be effective to appropriately align the nitrogen-containing aromatic bases 350, 360 in a stacked arrangement capable of chelating metal ions 420 and conducting electrons. For example, phosphoramide, phosphorothioate, phosphorodithioate, O-methylphosphoroamidite or peptide nucleic acid linkages may be effective to form such a backbone. Similarly, other components of the backbone 380 may vary, encompassing the deoxyribose moieties, ribose moieties, or combinations thereof, for example.

Alternatively, other types of bases may be substituted. For example, the nitrogen-containing aromatic bases 350 and 360 may be those that occur in native DNA and RNA, and thus, the nitrogen-containing aromatic bases may be selected from the group consisting of adenine, thymine, cytosine, guanine or uracil, or variants thereof such as 5-fluorouricil or 5-bromouracil. Alternative aromatic compounds may be utilized, such as aromatic compounds capable of interchelating a divalent metal ion coordinated to an atom in the aromatic compound, and capable of stacking, to produce a conductive metal-containing oligonucleotide duplex. Alternative aromatic compounds may for example include: 4-acetylcytidine; 5-(carboxyhydroxymethyl) uridine; 2′-O-methylcytidine; 5-carboxymethylaminomethyl-2-thiouridine; 5-carboxymethylaminomethyluridine; dihydrouridine; 2′-O-methylpseudouridine; beta, D-galactosylqueuosine; 2′-O-methylguanosine; inosine; N6-isopentenyladenosine; 1-methyladenosine; 1-methylpseudouridine; 1-methylguanosine; 1-methylinosine; 2,2-dimethylguanosine; 2-methyladenosine; 2-methylguanosine; 3-methylcytidine; 5-methylcytidine; N6-methyladenosine; 7-methylguanosine; 5-methylaminomethyluridine; 5-methoxyaminomethyl-2-thiouridine; beta, D-mannosylqueuosine; 5-methoxycarbonylmethyl-2-thiouridine; 5-methoxycarbonylmethyluridine; 5-methoxyuridine; 2-methylthio-N-6-isopentenyladenosine; N-((9-beta-D-ribofuranosyl-2-methylthiopurine-6-yl)carbamoyl)threonine; N-((9-beta-D-ribofuranosylpurine-6-yl) N-methycarbamoy1)threonine; uridine-5-oxyacetic acid-methylester; uridine-5-oxyacetic acid; pseudouridine; queuosine; 2-thiocytidine; 5-methyl-2-thiouridine; 2-thiouridine; 4-thiouridine; 5-methyluridine; N-((9-beta-D-ribofuranosylpurine-6-yl)-carbamoyl) threonine; 2′-O-methyl-5-methyluridine; and 2′-O-methyluridine; 3-(3-amino-3-carboxypropyl)uridine; hypoxanthine, 6-methyladenine, 5-me pyrimidines, particularly 5-methylcytosine (also referred to as 5-methyl-2′deoxycytosine and often referred to in the art as 5-me-C), 5-hydroxymethylcytosine (HMC), glycosyl HMC and gentobiosyl HMC, as well as synthetic nucleobases, e.g., 2-aminoadenine, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine.

In some embodiments, as for example illustrated in FIG. 2, the estimated spacing between the divalent metal ions 420 may be about 3, 4 or 5 Å (Angstroms).

The oligonucleotides may include those containing modified backbones, for example, phosphorothioates, phosphotriesters, methyl phosphonates, short chain alkyl or cycloalkyl intersugar linkages or short chain heteroatomic or heterocyclic intersugar linkages. In some embodiments, the phosphodiester backbone of the oligonucleotide may be replaced with a polyamide backbone, the nucleobases being bound directly or indirectly to the aza nitrogen atoms of the polyamide backbone (Nielsen et al., Science, 1991, 254, 1497). Oligonucleotides may also contain one or more substituted sugar moieties, such as moieties at the 2′ position: OH, SH, SCH₃, F, OCN, OCH₃ OCH₃, OCH₃ O(CH₂)ₙ, CH₃, O(CH₂)ₙ, NH₂ or O(CH₂)ₙ, CH₃ where n may for example be from 1 to about 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂ N₃; NH₂; heterocycloalkyl; heterocycloalkaryl; aminoalkylamino; polyalkylamino; substituted silyl; an RNA cleaving group; a reporter group; an intercalator; and other substituents having similar properties. Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group. Oligonucleotides may also include, additionally or alternatively, nucleobase (often referred to in the art simply as “base”) modifications or substitutions.

If desired, the divalent metal cations may be substituted for imine protons of the nitrogen-containing aromatic bases, and the nitrogen-containing aromatic bases are selected from the group consisting of thymine and guanine.

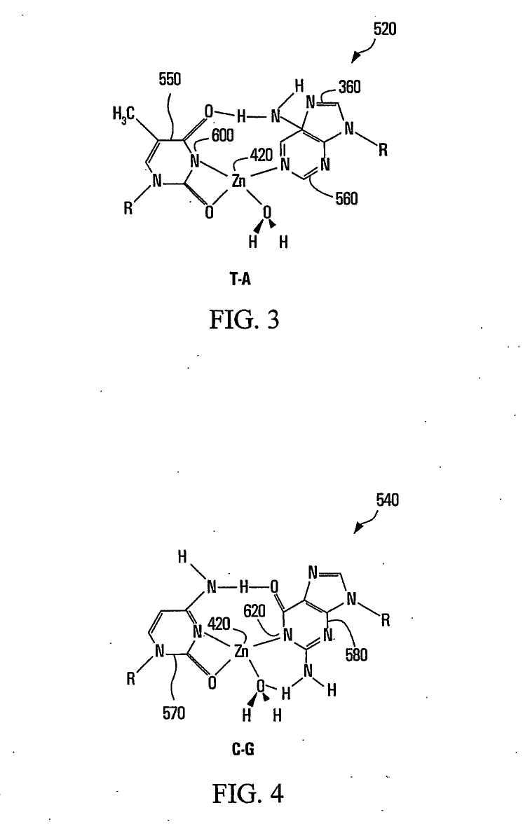

Referring to FIG. 3, a base-pairing scheme for the M-DNA 300 according to the present embodiment is shown generally at 520. In the base-pairing scheme 520, at least one of the nitrogen-containing aromatic bases includes thymine, having an N3 nitrogen atom, and the divalent metal cation is coordinated by the N3 nitrogen atom. More particularly, in this embodiment the base-pairing scheme 520 includes a thymine-adenine base pair, and the divalent metal cation 420 is zinc. Alternatively, the divalent metal cation 420 may be selected from the group consisting of zinc (Zn²⁺), cobalt (Co²⁺) and nickel (Ni²⁺). Alternatively, other divalent metal ions may be substituted depending upon the ability of the ions to participate with the other substituents in the formation of a conductive metal-containing oligonucleotide duplex. Alternatively, the metal cation may be selected from the group consisting of the cations of Li, Be, Na, Mg, Al, K, Ca, Sc, Ti, V, Cr, Mn, Fe, Co, Ni, Cu, Zn, Ga, Ge, As, Rb, Sr, Y, Zr, Nb, Mo, Tc, Ru, Rh, Pd, Ag, Cd, In, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, TI, Pb, Bi, Po, Fr, Ra, Ac, Th, Pa, U, Np and Pu. For example, in some embodiments, varying amounts of metal cations may be incorporated into a duplex, such as Zn²⁺, Ni²⁺, CO²⁺, Cd²⁺, Hg²⁺, Pt²⁺ and Ag¹⁺, where metal ions such as Cd²⁺, Hg²⁺, Pt²⁺ and Ag¹⁺ may constitute only a portion of the metal ions in the duplex, in effect ‘doping’ the duplex. The formation of a metal-substituted duplex using alternative cations under alternative conditions may be monitored, for example, using an ethidium bromide fluorescence assay.

In this embodiment, in the thymine-adenine base pair of the base-pairing scheme 520 shown in FIG. 3, one nitrogen-containing aromatic base is thymine 550 which possesses an N3 nitrogen atom 600. The divalent metal cation 420 (which in this embodiment is zinc) is coordinated by the N3 nitrogen atom 600 of the thymine 550, where the divalent metal cation zinc is substituted for an imine proton of the nitrogen-containing aromatic base.

Referring to FIG. 4, a base-pairing scheme for M-DNA according to a second embodiment of the invention is shown generally at 540. In the embodiment shown in FIG. 4, at least one of the nitrogen-containing aromatic bases includes guanine, having an N1 nitrogen atom, and the divalent metal cation is coordinated by the N1 nitrogen atom. More particularly, in this embodiment the base-pairing scheme 540 includes a cytosine-guanine base pair, in which one of the nitrogen-containing aromatic bases is guanine 580, which has an N1 nitrogen atom 620. As with the embodiment shown in FIG. 3, in this embodiment the divalent metal cation 420 is zinc. Alternatively, the divalent metal cation 420 may be selected from the group consisting of zinc (Zn²⁺), cobalt (Co²⁺) and nickel (Ni²⁺), or may include other suitable cations. In this embodiment, the divalent metal cation 420, which in this embodiment is zinc, is coordinated by the N1 nitrogen atom. Alternatively, the divalent metal cation 420 may be complexed between aromatic moieties in alternative conformations. In some embodiments, as illustrated, the imino protons of each base pair may be replaced by a metal ion.

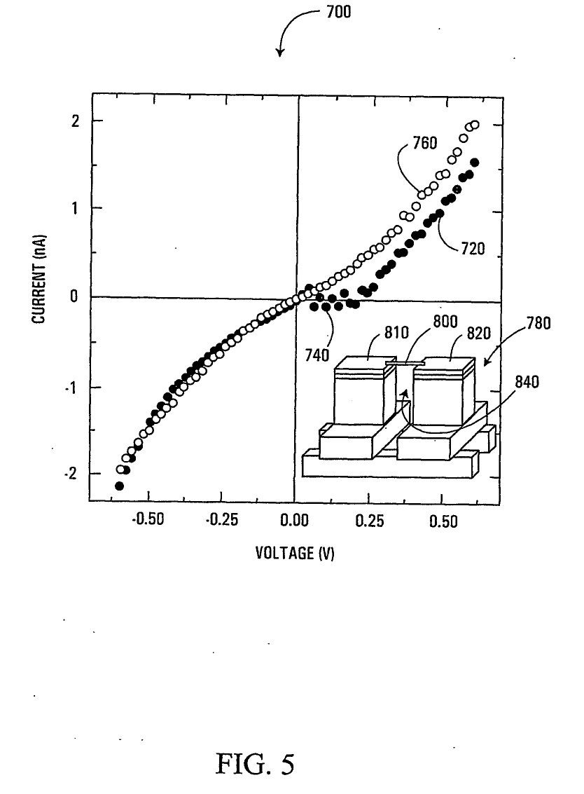

Referring to FIG. 5, the electrical (I-V) characteristics of an M-DNA may be measured as shown in FIG. 5, and as disclosed in Rakitin et al., 2000. For example, M-DNA may be prepared, such as the M-DNA prepared by Rakitin et al., from a B-DNA form of phage λ-DNA in 0.1 mM Zn²⁺ at a pH of 9.0, having sticky ends which can be utilized to bind each end in turn to an individual metallic electrode, such as a source electrode 810 and a drain electrode 820, which in this embodiment include gold electrodes (Braun et al., 1998).

A schematic testing layout to provide conductivity measurements of M-DNA is shown generally at 780 in the inset in FIG. 5. In this arrangement, a nucleic acid 800 is placed between the source electrode 810 and the drain electrode 820 separated by a deep physical gap 840, which may for example have a width of 1-30 microns.

Examples of I-V characteristics measured in vacuum (10⁻³ torr) at room temperature on samples of M-DNA and B-DNA are shown together generally at 700 in FIG. 5. A curve corresponding to B-DNA 720 shows a semiconductor like plateau (a band gap or conductance gap 740) of about 200 meV. In contrast, the I-V characteristic for M-DNA 760 shows no conductance gap. This is a characteristic difference between metallic and insulating behavior showing that electrons in M-DNA can conduct current down to extremely low voltages while B-DNA cannot. Thus, the qualitative difference in I-V characteristics of M-DNA 760 and B-DNA 720 at low bias voltages are indicative of a difference in their conduction mechanism.

In this embodiment, the M-DNA 300 is formed by annealing and treating a plurality of oligonucleotides to form a plurality of members, each member of the plurality of members including a pair of the oligonucleotides aligned to form a duplex portion. More particularly, in this embodiment the plurality of members include the donor member 104, the acceptor member 106, and the regulator member 108, and the annealing and treating of the plurality of oligonucleotides forms the members in a configuration in which the donor member, the acceptor member and the regulator member intersect to define the electric field regulation junction 112.

In the present embodiment, the oligonucleotides are annealed in conditions effective to form the duplex portion, and are treated in conditions effective to form the electric field regulation junction. More particularly, in this embodiment annealing includes subjecting the plurality of oligonucleotides to a basic solution under conditions effective to form the conductive metal-containing oligonucleotide duplex portion. In this embodiment, the conditions effective to form the conductive metal-containing oligonucleotide or nucleic acid duplex portion are effective to substitute the divalent metal cations for an imine proton of a nitrogen containing aromatic base in the conductive metal-containing oligonucleotide duplex portion. Thus, in this embodiment, producing the conductive metal-containing nucleic acid duplex includes subjecting the nucleic acid duplex to a basic solution in the presence of a metal cation (which in this embodiment is a divalent metal cation) under conditions effective to form the conductive metal-containing nucleic acid duplex, wherein the hydrogen-bonded base pairs of the conductive metal-containing nucleic acid duplex include an interchelated metal cation coordinated to a nitrogen atom in one of the nitrogen-containing aromatic bases. Similarly, in this embodiment, treating the plurality of oligonucleotides includes subjecting the nucleotides to the basic solution under conditions effective to form the electric field regulation junction. In the present embodiment, the basic solution has a pH of at least 7.

More generally, the conditions effective to form the M-DNA 300 will vary depending on the divalent metal cation 420 or ions used and the nature of the nucleic acid strands 320 and 340. Routine assays may be carried out to determine appropriate conditions effective for conductive duplex formation, for example by varying parameters such as pH, nucleic acid concentration, metal ion concentration, and the ratio of the metal ion concentration to the nucleic acid concentration. In some embodiments, a pH equal to or greater than 7, 7.5, 8, 8.5 or 9 may be desirable, and a suitable nucleic acid to metal ion ratio may be from about 1:1.5 to about 1:2.0, for example.

In some embodiments, M-DNA 300 may be formed from B-DNA by the addition of metal ions, such as 0.1 mM Zn²⁺ or mM NiCl₂ at an approximate pH, such as a pH of 9.0. There may be a concomitant release of protons, so that a base such as KOH may be added to maintain the pH at a desired level, such as at 8.

As is evidenced by the conductive behaviour shown in FIG. 5, configurations of conductive M-DNA may provide switching functionality of current and/or voltage to regulate electronic signals.

Referring back to FIG. 1, in this embodiment the three arms 120, 140 and 160 intersecting to define the conductive junction 180 enable the organic circuit element 100 to function as an electric signal regulator. Three-way junctions such as the conductive junction 1180 may for example be prepared from three strands of oligonucleotides 1140, 1160 and 1180, each having 5′ and 3′ ends, the sequences of which may be chosen so that they can only anneal in the desired configuration. In the embodiment shown in FIG. 1, the three-way conductive junction 180 was constructed from the three strands of oligonucleotides 1140, 1160 and 1180, which in this embodiment include three 60-mer oligonucleotides, forming duplex portions (namely, the modulator arm 120, the acceptor arm 140, and the donor arm 160) out of pairs of antiparallel oligonucleotides.

Still referring to FIG. 1, in this embodiment, the electron donor 200 includes a first electrode 202 operable to donate an electron to the donor member 104, and the electron acceptor 220 includes a second electrode 222 operable to accept an electron from the acceptor member 106. Also in this embodiment, the electric field regulator 114, or more particularly the electron flow modulator 240, includes a third electrode 242. If desired, the third electrode may be operated to accept an electron from the acceptor member or to donate an electron to the donor member. The electrodes 202, 222 and 242 may include gold electrodes, for example. Gold electrodes may for example be attached to DNA by incorporating a thiol at the 5′ end in place of the chromophore (Wang et al., 1999). A current or voltage may be externally applied to the organic circuit element 100 across the donor arm 160 and the acceptor arm 140.

Alternatively, the electron donor, electron acceptor and electric field regulator need not include electrodes.

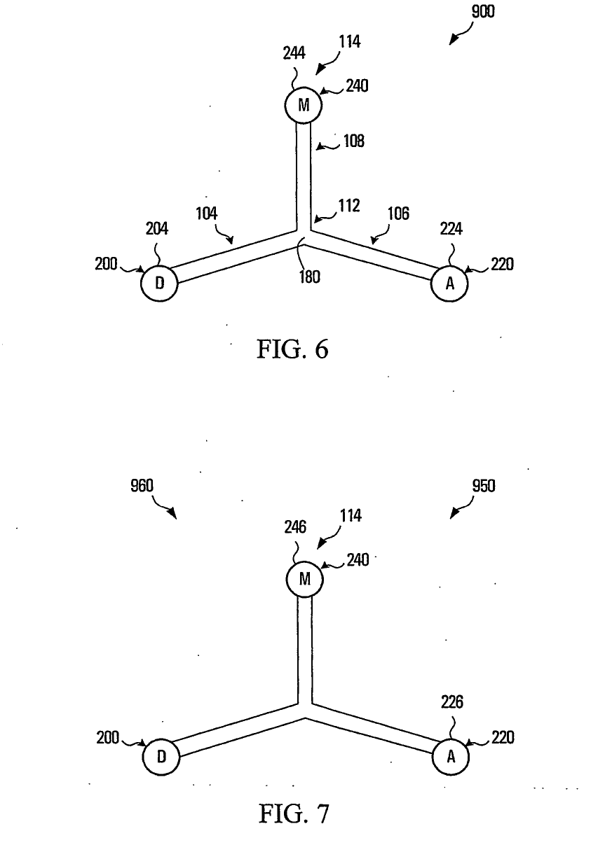

For example, referring to FIGS. 1 and 6, an organic circuit element according to a third embodiment of the invention is shown generally at 900 in FIG. 6. The organic circuit element 900 is generally similar to the organic circuit element 100 shown in FIG. 1, however, in the embodiment shown in FIG. 6, the electron donor 200 of the organic circuit element 900 includes an electron donor molecule 204 capable of donating an electron to the donor member 104 (which in this embodiment includes the donor arm 160). In the present embodiment the electron donor molecule 204 includes a fluorescent molecule, or more particularly, a fluorescein. Similarly, the electron acceptor 220 of the organic circuit element 900 includes an electron acceptor molecule 224 capable of accepting an electron from the acceptor member 106 (which in this embodiment includes the acceptor arm 140). In the present embodiment, the electron acceptor molecule 224 also includes a fluorescent molecule, or more particularly, a rhodamine. Also in this embodiment, the electric field regulator 114, or more particularly the electron flow modulator 240, includes a regulator molecule 244 selected from the group consisting of fluorescent molecules and chromophores. Thus, in this embodiment, the states of the electric field regulator 114 may be selected by irradiating the electric field regulator. More particularly, in this embodiment the regulator molecule 244 includes a fluorescent molecule, such as a fluorescein or a rhodamine, for example. Alternatively, other suitable regulator molecules may be substituted.

Similarly, referring to FIGS. 1 and 7, an organic circuit element according to a fourth embodiment of the invention is shown generally at 950 in FIG. 7. In this embodiment, the electric field regulator 114, or more particularly, the electron flow modulator 240, includes a regulator or modulator chromophore 246, which in this embodiment absorbs radiation within a range of wavelengths. Thus, the states of the electric field regulator 114 may be selected by irradiating the electric field regulator. In this embodiment, irradiating the modulator chromophore 246 causes a negative electrostatic potential to be applied to the electric field regulation junction 112, the negative electrostatic potential decreasing the ability of an electron to travel from the donor member 104 to the acceptor member 106. Similarly, in this embodiment the electron acceptor 220 includes a chromophore 226 operable to emit radiation within a range of wavelengths in response to accepting an electron from the acceptor member 106.

Similarly, in other embodiments, the electric field regulator 114, the electron donor 200 and the electron acceptor 220 may include any other suitable combinations or permutations of electrodes, fluorescent molecules, chromophores, or other suitable molecules. In this regard, fluorescent molecules and electrodes may be particularly useful in combination for some applications of embodiments of the present invention, due to the ability of fluorescent molecules to generate photocurrents when irradiated and subjected to an applied potential. For example, it has been found that fluorescein-labelled M-DNA assembled on a gold electrode and subjected to an applied potential of 0.2 volts generates an appreciable photocurrent of approximately 0.03 mA when the fluorescein is irradiated, but does not generate any appreciable photocurrent when the fluorescein is not being irradiated. (At higher potentials, however, some current may be observed regardless of irradiation, due to electrolysis.) Similarly, irradiation of chromophore-labelled M-DNA attached to a gold electrode also produces an appreciable current.

In some such exemplary embodiments, the 5′ end of each arm 120, 140 and 160 was attached either to fluorescein, rhodamine or a control, not labeled. As used herein, a nomenclature for labeled circuit elements may be based on identifying each arm 120, 140 and 160 with a letter (F, R, or C) to specify whether that arm contains, respectively, fluorescein (F), rhodamine (R) or a control (C, no label). Thus, for example, 160F:120C:140R-60 represents three 60-mer oligonucleotide strands 1140, 1160 and 1180 assembled to form the conductive junction 180, where fluorescein is the electron donor 200 attached to the donor arm 160, rhodamine is the electron acceptor 220 connected to the acceptor arm 140, and the electron flow modulator 240 is absent and therefore not connected to the modulator arm 120.

The fluorescence of the electron donor 200 of the organic circuit element 100 may then be measured by fluorescence assay to confirm the conductivity of the junction 180. During such an assay, the fluorescence will be quenched if there is electron transfer along the M-DNA, through the junction. If, on the other hand, there is little conduction along the donor arm 160 and the acceptor arm 140 (as would be the case if these arms had been formed of B-DNA rather than M-DNA for example), the fluorescence of the electron donor 200 will not be quenched to the same degree. In one such exemplary embodiment, the fluorescence of fluorescein acting as the electron donor 200 was measured for M-DNA 160F:120C:140R-60 and compared to another exemplary embodiment, 160F:120C:140C-60, which has the same configuration except that the latter embodiment does not include rhodamine acting as the electron acceptor 220 connected to the acceptor arm 140. The fluorescein fluorescence was 40% quenched for the former embodiment (160F:120C:140R-60) compared to the latter embodiment (160F:120C:140C-60), confirming that electrons are transferred from the fluorescein electron donor 200 through the donor arm 160 and the conductive junction 180 to the acceptor arm 140 and the rhodamine electron acceptor 220.

Other such exemplary embodiments employing a fluorescent molecule as the electron donor 200 may be similarly used to confirm the ability of the electric field regulator 114 to regulate the electric field at the electric field regulation junction 112. For example, two exemplary embodiments, 160F:120R:140R-60 and 160F:120F:140R-60, having a rhodamine or a fluorescein as the electron flow modulator 240 connected to the modulator arm 120, were separately compared to a control sample, 160F:120C:140R-60. During respective fluorescence assays, the fluorescein fluorescence was quenched by 60% (160F:120R:140R-60) and 35% (160F:120F:140R-60) relative to the control sample. Therefore an electron donor or acceptor, such as fluorescein or rhodamine, attached to the modulator arm 120 can alter the conductivity between the donor arm 160 through the conductive junction 180 and to the acceptor arm 140. Thus, the circuit element 100 may act as a switch having alternative states.

More generally, referring to FIGS. 1, 6 and 7, any of the organic circuit elements 100, 900 and 950 (or the other organic circuit elements described in greater detail below, for example) may be used to regulate an electronic signal between first and second locations in a conductive nucleic acid material. In this embodiment, the first location; may include the electron donor 200, or alternatively, may be considered to include any location on the donor member 104 between the electron donor 200 and the electric field regulation junction 112. Similarly, in this embodiment the second location may include the electron acceptor 220, or any location on the acceptor member 106 between the electron acceptor 220 and the electric field regulation junction 112. The electronic signal itself may be produced by causing electrons to flow from the first location to the second location, in any suitable way, such as by applying a voltage between the electron donor and the electron acceptor, irradiating the donor and acceptor, and/or supplying electrons to the first location and receiving electrons from the second location.

The regulation of the electronic signal between the first and second locations may be achieved by varying an electrostatic potential at a third location in the nucleic acid material interposed between the first and second locations. In the embodiments shown in FIGS. 1, 6 and 7, the third location includes the electric field regulation junction 112. The varying of the electrostatic potential may be achieved by selecting one of the plurality of states of the electric field regulator 114, which is in communication with the third location, each of the states corresponding to a respective electrostatic potential at the third location. In the case of the organic circuit elements 900 and 950 shown in FIGS. 6 and 7, selecting one of the states may be achieved by irradiating the electric field regulator. This may cause a negative electrostatic potential to be applied to the third location, for example. In the case of the organic circuit element 100 shown in FIG. 1, selecting one of the states may be achieved by applying an external potential to the electric field regulator 114, or more particularly, to the electrode 242. This may include depositing at least one electron onto the electrode 242 to apply a negative electrostatic potential to the third location, or alternatively, removing at least one electron from the electrode 242 to apply a positive electrostatic potential to the third location. A negative electrostatic potential at the electric field regulation junction 112 tends to decrease the ability of an electron to travel from the donor member to the acceptor member, while a positive electrostatic potential at the junction tends to increase its ability to do so. Thus, any of the circuit elements shown in FIGS. 1, 6 and 7 acts as an apparatus for regulating an electronic signal between first and second locations in a conductive nucleic acid material, the apparatus including an electric field regulator operable to vary an electrostatic potential at a third location in the nucleic acid material interposed between the first and second locations.

Referring back to FIG. 7, in alternative embodiments, a modulator chromophore 246 may be selected as the electric field regulator 114, so that it absorbs irradiation at a wavelength that is different from the wavelengths at which both the electron donor and the electron acceptor, such as fluorescein and rhodamine, absorb irradiation. Upon selective irradiation of the modulator chromophore 246, an electron is excited to a higher energy state on the modulator chromophore which thus produces a change in the conductivity or electrostatic potential (voltage) at the conductive junction 180. In some embodiments, a negative electrostatic potential may be established at the conductive junction 180 which may impede conductivity or the passage of electrons through the conductive junction 180. After some time, the modulator chromophore 246 may return to a different state, for example an excited electron in the chromophore 246 may emit a photon and fall back into its ground state, thus returning the electrostatic potential or conductivity at the conductive junction 180 to its original value (or a further alternative value). In this way, the conductive junction 180 may act as a gate to regulate the flow of signals or electrons from the donor arm 160 to the acceptor arm 140. In one embodiment, for example, the conductive junction 180 may act as a gate switch which may be in an “on” state when the modulator chromophore is un-irradiated and thus allows electrons or a signal to flow from the donor arm 160 to the acceptor arm 140, and the gate may be in an “off” state when the modulator chromophore 246 is irradiated and its electron is excited to a higher energy state. Thus, in such embodiments, the organic circuit element 100 behaves in some ways analogously to a field effect transistor in which the electron donor 200 acts as a source electrode, the electron acceptor 220 acts as a drain electrode, and the electric field regulator 114 (such as the modulator chromophore 246) acts as a gate electrode. The electric field regulator 114, acting as a gate electrode, may act to control the effective electron diameter of a channel of electron flow flowing from the donor arm 160 through the conductive junction 180 to the acceptor arm 140. Effectively, the flow of electrons from the electron donor 200 (source electrode) is controlled by the voltage or change in electrostatic potential applied by the electric field regulator 114 to the conductive junction 180. The voltage applied to the conductive junction (gate) may be regulated or modulated by the electron flow modulator 240 and by the modulator arm 120. By regulating the “on” and “off” state of the “gate switch” in this manner, to vary the electrostatic potential at the conductive junction 180, the organic circuit element 100 may be used to create, store and erase memory by representing zeros and ones in the alternative states.

Thus, referring to FIG. 7 for example, an organic data storage medium is shown generally at 960. The storage medium 960 includes the electric field regulator 114, which has at least two selectable states, each of the states corresponding to a respective degree of electric field regulation at an electric field regulation junction of a nucleic acid circuit element, each degree of electric field regulation corresponding to a respective data value. In this embodiment, the organic data storage medium 960 further includes the organic nucleic acid circuit element 950, which in turn includes the donor member 104, the acceptor member 106, and the regulator member 108 intersecting with at least one of the plurality of members (in this embodiment, intersecting both the donor member and the acceptor member) to define the electric field regulation junction 112, for cooperating with the electric field regulator 114 to apply the degree of electric field regulation to the junction, to represent the data value.

In this embodiment, each of the at least two states of the electric field regulator corresponds to a respective electrostatic potential at the electric field regulation junction.

In the present embodiment, the at least two states are selectable by irradiating the electric field regulator. More particularly, in this embodiment, the at least two selectable states include an excited state and a ground state of the chromophore 246. The chromophore 246 may be maintained in an excited state by irradiating it, to represent a data value such as a binary “1”, for example, and may be allowed to revert to its ground state by ceasing such irradiation, to represent a data value such as a binary “0”, for example. As discussed above, when the chromophore is in the excited state, the electrostatic potential at the electric field regulation junction 112 is altered or varied, thereby altering the conductivity at the conductive junction 180. The data value so stored may then be “read” in any suitable way. For example, an external potential may be applied between the electron donor 200 and the electron acceptor 220, and the resulting current may be measured, a first measured current value being indicative of the excited state representing a binary “1”, a second measured current value being indicative of the ground state representing a binary “0”.

Referring back to FIG. 1, an alternative organic data storage medium may include the organic circuit element 100, in which the at least two states are selectable by applying an external potential to the electric field regulator 114, which in the embodiment shown in FIG. 1 includes the electrode 242.

More generally, however, many useful applications other than data storage exist for such methods of regulating an electronic signal in a conductive nucleic acid material by varying a degree of electric field regulation at an electric field regulation junction, as described above.

Referring back to FIG. 1, a system may be provided, the system including the organic circuit element 100 and further including a conductive medium 1190 for supplying conduction electrons to the electron donor 200 and for receiving conduction electrons from the electron acceptor 220. In some such embodiments, a current may flow when an organic circuit element such as the circuit element 100 is included in the conductive medium 1190. The conductive medium 1190 may be any medium which is operable to donate electrons to the electron donor 200 and accept electrons from the electron acceptor 220 to provide a closed circuit way for electrons to flow from the electron donor 200, through the donor member 104 (in this embodiment, the donor arm 160), through the electric field regulation junction 112 (which in this embodiment includes the conductive junction 180), through the acceptor member 106 (which in this embodiment includes the acceptor arm 140), through the electron acceptor 220, and back to the electron donor. The conductive medium 1190 may include an aqueous solution, for example, to provide conduction between the electron donor 200 and the electron acceptor 220. Alternatively, the conductive medium 1190 may include a conductive wire, for example, or any other suitable conductive medium may be substituted.

Referring back to FIG. 1, in alternative embodiments, not all of the plurality 102 of members necessarily include a conductive metal-containing oligonucleotide duplex. More particularly, one or more of the arms 120, 140 or 160 may not form a conductive duplex under conditions where one or more of the remaining arms, 120, 140 or 160 does form a conductive duplex. In one such embodiment, the donor member 104 and the acceptor member 106 may include such a conductive metal-containing oligonucleotide duplex, while one or more other members do not. For example, the modulator arm 120 may have a composition which will not form a conductive duplex when the donor arm 160 and the acceptor arm 140 do form a conductive duplex. In this way, combinations of B-DNA and M-DNA may be used for portions of the arms 120, 140 or 160. For example, duplexes containing 5-fluorouricil may form M-DNA while duplexes lacking this base may not, so that the composition of nucleic acid strands 1140, 1160 and 1180 may be adapted so that the donor arm 160 and the acceptor arm 140 contain a high proportion of 5-fluorouricil. In this way, the effect of the modulator 240 on the conductive junction 180 may be made dependent upon the conditions to which element 100 is subjected (dictating whether an arm is in the form of B-DNA or M-DNA). Similarly, nucleic acid binding proteins may be used to modulate conductivity of the arms 120, 140 and 160.

In alternative embodiments, the electron flow modulator 240 may be capable of absorbing or donating electrons from a conductive medium, while being electrically insulated from the conductive junction 180 by a non-conductive modulator arm 120. A non-conductive modulator arm 120 may for example be formed, as described above, under conditions wherein a conductive duplex is formed on the donor arm 160 and the acceptor arm 140, but not on the modulator arm 120.

In alternative embodiments, the organic circuit element 100 may be constructed to provide different forms of functionality. The electron acceptor 220 may, for example, act as a detectable label for conductivity of the circuit element 100. For example, the electron acceptor 220 may be a chromophore, which upon accepting an electron, may emit a photon at a different or characteristic wavelength, so that the emitted photon may be detected.

In alternative embodiments, organic circuit elements may include a plurality of donor arms, acceptor arms, or modulator arms.

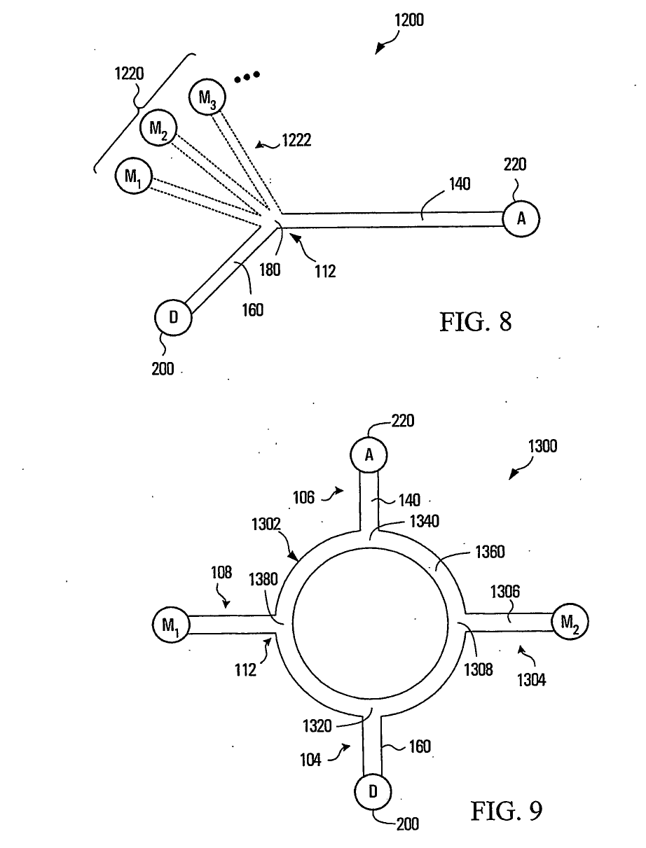

For example, referring to FIG. 8, an organic circuit element according to a fifth embodiment of the invention is shown generally at 1200. In this embodiment, the plurality 102 of members includes a plurality 1220 of regulator members, formed in a configuration in which the plurality 1220 of regulator members intersects the plurality 102 of members to define the at least one electric field regulation junction 112. More particularly, in this embodiment, the organic circuit element 1200 includes the donor arm 160 and the acceptor arm 140, both of which intersect at a conductive junction 180 with a plurality 1222 of electron flow modulator arms, which in turn are connected to respective electron flow modulators. The strands of oligonucleotides used to form the organic circuit element 1200 may be chosen in the appropriate sequences so that they can only anneal in the desired configuration, each strand of oligonucleotides forming the duplexes which make up the modulator arms 1222, the donor arm strand 1160 and the acceptor arm strand 1140 typically being aligned anti-parallel. Advantageously, separate electron flow modulators M₁, M₂, M₃, . . . may be used which are each separately responsive to a different condition or signal, such as a particular wavelength of light. In this way, the organic circuit element 1200 may be used as a detector to detect a particular signal, such as a signal or condition inside biological systems.

Referring to FIG. 9, an organic circuit element according to a sixth embodiment of the invention is shown generally at 1300. In this embodiment, the plurality 102 of members includes a common member 1302, which in this embodiment includes a circular DNA portion 1360. In the present embodiment, the donor member 104, the acceptor member 106 and the regulator member 108 intersect the common member 1302 at first, second and third locations (or junctions) 1320, 1340 and 1380 respectively, the third location 1380 defining the electric field regulation junction 112. Thus, in this embodiment, the donor arm 160 and the acceptor arm 140 are connected at separate locations or junctions 1320 and 1340 respectively to the circular DNA portion 1360. Also in this embodiment, a second regulator member 1304, which in this embodiment includes a second modulator arm 1306, intersects the common member 1302 at a fourth location 1308 defining a second electric field regulation junction. Thus, in this embodiment the organic circuit element 1300 includes multiple junctions at the locations 1380 and 1308 connecting to multiple respective electron flow modulators M₁ and M₂ which may be the same or different. Thus, in this embodiment the at least one electric field regulation junction includes at least two electric field regulation junctions (at the locations 1308 and 1380) in electrical communication with at least two respective electric field regulators, and regulation or modulation may be achieved by selecting one of a plurality of states of at least one of the two electric field regulators, each of the states corresponding to a respective electrostatic potential at the electric field regulation junction corresponding to the at least one of the two regulators.

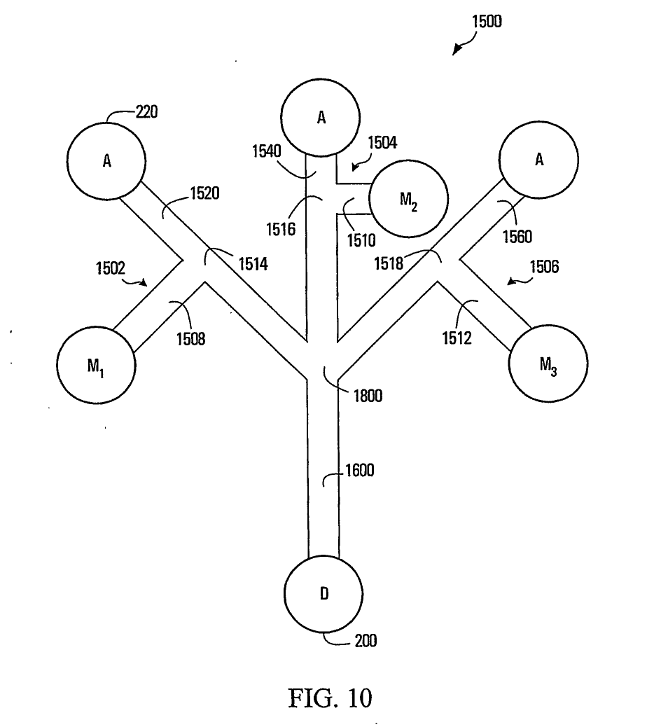

An organic circuit element according to a seventh embodiment is shown generally at 1500 in FIG. 10. In this embodiment, the at least one regulator member includes a plurality of regulator members, which intersect other respective members of the plurality 102 of members to define a plurality of respective electric field regulation junctions. In this embodiment, each such regulator member intersects with one of the donor member and the acceptor member to define the electric field regulation junction, rather than intersecting with both the donor member and the acceptor member. More particularly, in this embodiment the organic circuit element 1500 includes first, second and third regulator members 1502, 1504 and 1506, which in turn include respective modulator arms 1508, 1510 and 1512. In this embodiment, the modulator arms 1508, 1510 and 1512 intersect with respective acceptor arms 1520, 1540 and 1560 to define respective electric field regulation junctions 1514, 1516 and 1518. The acceptor arms 1520, 1540 and 1560 intersect each other and intersect an electron donor arm 160 to define a conductive junction 1800. Thus, the organic circuit element 1500 includes multiple electron flow modulators M₁, M₂, M₃ and electron flow-modulator arms 1508, 1510 and 1512 connected to each acceptor arm of the plurality of acceptor arms. It will be appreciated that variations in electrostatic potential at any of the electric field regulation junctions 1514, 1516 and 1518 will also result in electrostatic potential variations at the conductive junction 1800, which therefore also effectively acts as an electric field regulation junction.

It is noted that organic circuit elements according to some embodiments of the invention may be used to detect the presence of a particular nucleic acid homologous to a single stranded component of an electron modulator arm. Nucleic acid in a sample may for example be labeled to include an electron flow modulator, such as fluorescein, and the sample may be mixed with organic circuit elements having single stranded electron modulator arms, so that if a nucleic acid is present in the sample that is homologous to the single stranded modulator arm, it will hybridize. Following hybridization, conditions may be adjusted to favor the formation of a conductive duplex in the electron modulator arm, to bring the label attached to the sample nucleic acid into electrical communication with the remainder of the organic circuit element. The presence of the conductive electron modulator arm in the circuit element may be detected by a change in the conductivity between the electron door arm and the electron acceptor arm.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes, but is not limited to, all embodiments and variations substantially as hereinbefore described and with reference to the examples and drawings. More generally, while specific embodiments of the invention have been described and illustrated, such embodiments should be considered illustrative of the invention only and not as limiting the invention as construed in accordance with the accompanying claims.

### EXAMPLE

Abstract: In this example, an M-DNA complex was formed between duplex DNA and divalent metal ions at approximately pH 8.5. 30 base pair linear duplexes were prepared with fluorescein attached to on end, and anthraquinone at the other. Quenching of the fluorescence emission from fluorescein by anthraquinone was under conditions corresponding to M-DNA, but not for B-DNA. The quenching, which is ascribed to an electron transfer process, was blocked by chemical reduction using NaBH₄ of anthraquinone to the dihydroanthraquinone which is not an electron acceptor. Upon the reoxidation of the dihydroquinone by exposure to oxygen the quenching was restored. Quenching of fluorescein fluorescence was also observed in a 90 base pair Y-branched duplex in which rhodamine or anthraquinone were attached to one of two of the remaining arms. Thus the electron transfer process is not impeded by the presence of a junction in the duplex, contrary to results previously reported for B-DNA samples. Again the fluorescein fluorescence could be modulated by reduction of the anthraquinone group in the Y-branched duplexes, mimicking a simple chemical switch. Therefore M-DNA may have extraordinary potential for the development of nanoelectronic devices.

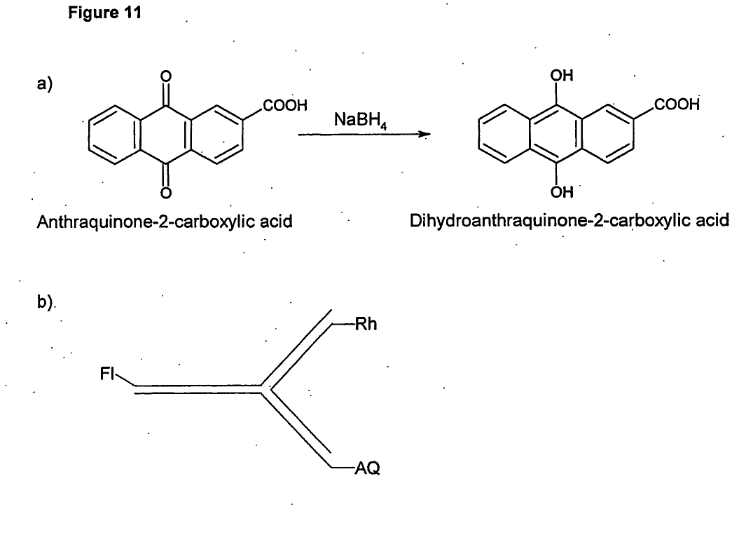

Detailed Description: Dyes such as anthraquinone(15,26-28) (and derivatives thereof) have been extensively used to probe charge transfer processes occurring in DNA, with the dye (in its excited state) serving as an electron acceptor from guanine; however, they have not been studied in donor/acceptor combinations separated by a DNA duplex. These dyes, and related biologically important quinones, are of particular interest in light of their intimate involvement in electron transport and in the photosynthetic pathway, and are being studied in the development of photosynthetic mimics(29). Here we report the results of a study of fluorescence quenching of fluorescein by anthraquinone in M-DNA using 30-base pair linear duplexes. Anthraquinone in observed to quench the fluorescence of fluorescein under M-DNA conditions for both structures; however, upon reduction of the anthraquinone dye to the hydroquinone (FIG. 11a), quenching is significantly reduced. In effect the electron transfer process is blocked by chemical reduction of the acceptor group.

Branched duplexes can be constructed from 3 distinct single strands having appropriate complementary sections, as shown in FIG. 11b. Previous studies have shown the branched duplexes to be a Y-shaped molecule with three arms in an essentially planar geometry with equal angles between each arm(30,31). The addition of metal cations does not result in helix-helix stacking observed in 4-way junctions, rather the 3-way junction remains in an extended y-shaped conformation(30,31). Such junctions, in B-DNA, typically results in less efficient electron transfer(32-34). Here, efficient electron transfer is observed to occur between fluorescein and the acceptors anthraquinone and/or rhodamine, through a Y-branched junction.

Materials and Methods: Fluorescence measurements were initially carried out using a 30 base pair sequence, in order to evaluate the efficiency of anthraquinone (AQ) as a quencher for fluorescein. Three 60 base single-strands were used to form a duplex, of a 90 base pair overall size, containing a Y-junction (see below), allowing for a number of donor-acceptor combinations. The sequences used in this study are given in Table 1. The y-junctions were prepared by incubating the three single strands in the dark, in 10 mM Tris-HCl (pH 8) and 10 mM NaCl at 65° C. for two hours, followed by slow cooling to room temperature(31). Agarose gel (4%) electrophoresis of the Y-branched duplexes demonstrated the formation of a single species with a mobility corresponding to 110-124 base pairs (data not shown). This is in agreement with previous reports, and suggests that the Y-shaped structure retards the migration of the duplex. (31)

Donor strands were labeled in the 5′ position with 5-carboxyfluorescein (FI) and the complementary (acceptor) strands were labeled in the 5′ position with 2-anthraquinonecarboxylic acid (Aldrich) or 5(6)-carboxytetramethyrhodamine (Rh). The dye molecules were covalently attached using a standard 6-aminohexyl linker. Where necessary, carboxylic acid derivatives were converted to activated esters prior to attachment. Sequences were obtained from either Calgary Regional DNA Synthesis Facility or from the DNA/Peptide Synthesis Lab at the National Research Council Plant Biotechnology Institute (Saskatoon). Fluorescence measurements were carried out using a Hitachi model F2500 fluorometer at DNA concentrations of 1.5 μM (in bases), unless otherwise specified, in 20 mM Tris-HCl buffer at either pH 7.5 for B-DNA conditions or pH 8.5 for M-DNA conditions. Fluorescein was excited at 490 nm, and the emission spectra recorded from 500-800 nm. Conversion to M-DNA was accomplished by the addition of 20 mM ZnCl₂ stock solution, to a final concentration of 0.2 mM(24).

The reduction of AQ was carried out using a 0.5 mM stock solution of NaBH₄ (made fresh prior to reduction)(35). Briefly, the NaBH₄ stock solution was added to a solution of 150 μM (in bases) AQ-labeled single stranded DNA, and incubated at room temperature for 2 hours. The reduced strand was then hybridized with the complementary fluorescein-labeled single strand to produce the fluorescein/dihydroanthraquinone labeled duplex. As a control experiment, both the fluorescein labeled single strand, as well as a fluorescein/anthraquinone duplex were also subjected to the same reduction process. Where necessary, samples were de-oxygenated by bubbling with nitrogen gas for a minimum of 30 minutes.

In order to ensure that the above procedure resulted in a reduction of the AQ group, the same procedure was carried out using 34 μM 2-anthraquinone N-hydroxysuccinimidyl ester (AQ-NHS) in pH 8.0 10 mM Tris-HCl, 10 mM NaCl buffer. This solution was degassed by bubbling with nitrogen for ½ hour prior to reduction. The reduction was carried out using 0.5 M NaBH₄, to a final concentration of 1.9 mM. UV-vis absorbance spectra were measured before and after the reduction procedure with a Gilford 600 spectrometer. Finally, in order to determine whether or not the reduction procedure results in damage to the strands themselves, polyacrylamide gel electrophoresis (PAGE) analysis of the reduced FI-30-AQ duplexes was carried out using a 20% polyacrylamide gel.

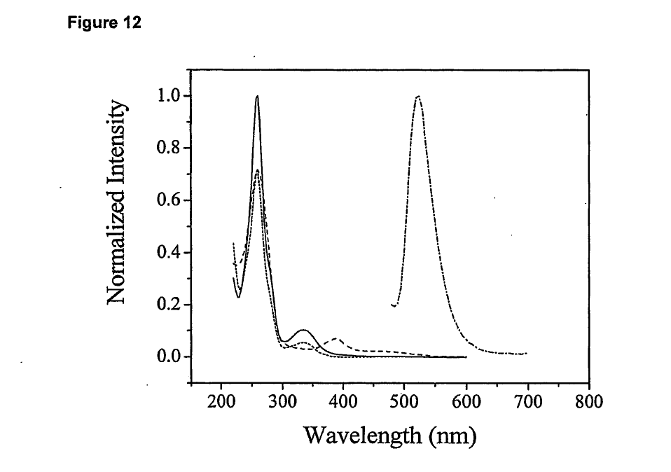

Results and Discussion: The absorbance spectra of AQ-NHS (in deoxygenated buffer solution) prior to and following reduction, and upon reoxidation are shown in FIG. 12. Upon addition of 3 mM NaBH₄ the characteristic absorption at 335 nm disappears with a new absorption at 388 nm, which corresponds to the hydroquinone(35). As anticipated, the hydroquinone could be reoxidized to the anthraquinone upon exposure to oxygen(36). Unfortunately, due to the high concentration of DNA required, it was not possible to carry out a similar experiment using the AQ-labeled DNA. However it is expected that the reduction will not be impacted by attachment to DNA.

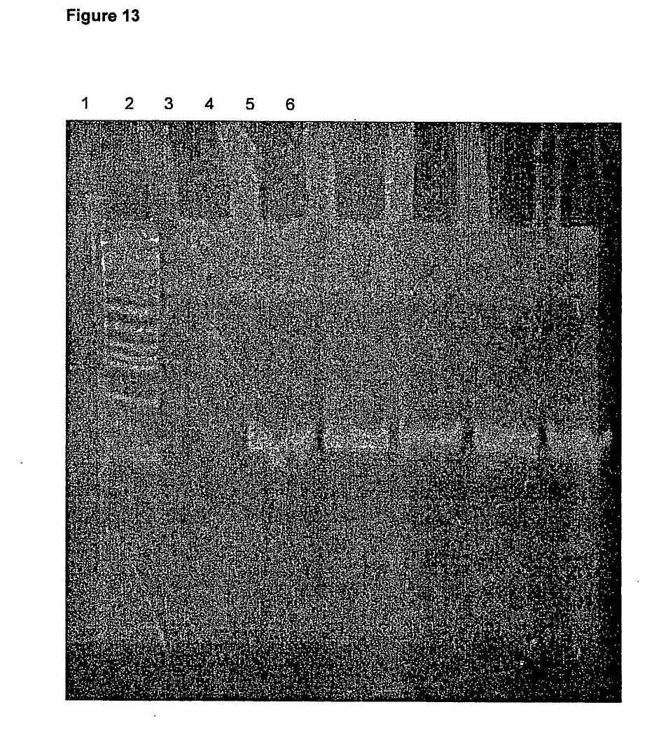

A number of control experiments were carried out in order to ensure that the addition of NaBH₄ did not result in either damage to the DNA. FIG. 13 illustrates the results of PAGE analysis of both the reduced and native FI-30-Aq duplexes. In all cases the migration of the FI-30-Aq duplex compares well with the corresponding DNA markers. By comparing lane 5 (0 mM NaBH₄) to lanes 3 and 4 (2.5 and 25 mM NaBH₄, respectively) of the gel it can be seen that the reduction procedure does not result in any damage to the labeled single strand; specifically, the untreated and treated duplexes migrate to the same level. Further, comparing lanes 3 and 6, it can be seen that reduction of the anthraquinone label following hybridization (lane 6) as opposed to prior to hybridization (lane 3) also does not result in any damage to the duplex itself. Similarly, an ethidium bromide fluorescence assay showed binding of ethidium to the treated duplex at the same level as untreated DNA. Any damage to the duplex would result in a loss of fluorescence due to decreased binding, which was not observed. Finally, fluorescence excitation and emission spectra for fluorescein and rhodamine remain unchanged upon treatment with NaBH₄, indicating that the addition of NaBH₄ did not result in their reduction.

The attachment of the anthraquinone group to a fluorescein-labeled 30-mer results in significant quenching of the fluorescein fluorescence upon formation of M-DNA, as shown in Table 2. Under the standard conditions used to form M-DNA(24), namely 0.2 mM Zn²⁺ concentration, rhodamine and anthraquinone quench the fluorescence from fluorescein to 86% and 59%, respectively. As observed previously(24,37) the degree of quenching depends on the nature of the acceptor and, as will be seen below, the length of the duplex. Due to the lack of spectral overlap (see FIG. 12) between fluorescein and anthraquinone, resonance energy transfer is not a possible mechanism for the de-activation of excited state fluorescein.(38) Further, in considering the redox potentials of fluorescein as an electron donor (E°_{Ox}=0.96 V(39), ΔE_{0,0}=2.46 eV) and anthraquinone as an electron acceptor (E°_{Red}=−0.94 V)(40), the Rehm-Weller equation(41) predicts an exergonically favorable electron transfer process with ΔG=−0.56 eV. Indeed, photo-induced electron transfer from fluorescein to anthraquinone (in molecular dyads) has previously been observed using both fluorescence quenching and ESR methods(42). In this dyad, anthraquinone was observed to quench the fluorescence from fluorescein by 98%, attributed to an electron transfer process with k_{ET}=4×10⁹ s⁻¹. Therefore, chemical reduction of anthraquinone in the M-DNA systems should result in a decrease in the fluorescence quenching of fluorescein, as it will no longer be able to accept an electron transferred from fluorescein.

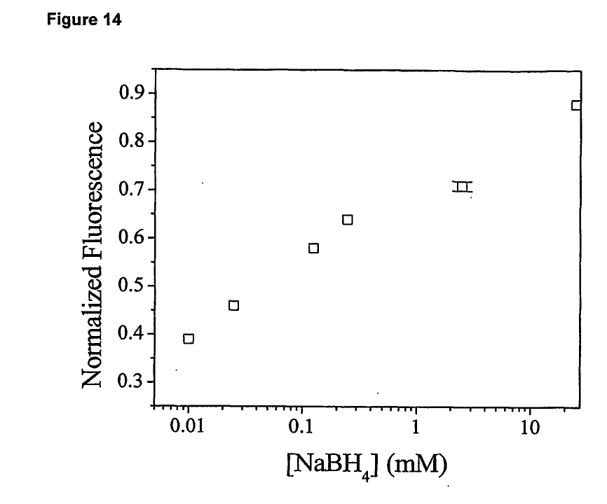

FIG. 14 shows that this is indeed the case, with the normalized intensity from fluorescein increasing, with increasing borohydride concentration. As a control, duplexes labeled with fluorescein (FI-30), and with both fluorescein and rhodamine (FI-30-Rh) were treated in the same manner as the FI-30-AQ duplex. As shown in Table 2, for the FI-30 duplex, no effect was observed, and the emission from fluorescein was unchanged. Similarly, the observed quenching for the FI-30-Rh duplex was also found to be unchanged by the reduction procedure. The results given in Table 2 show that the reduction of AQ by 2.5 mM NaBH₄ results in an increase in the normalized fluorescence from 0.41±0.04 for unreduced FI-30-AQ to 0.71±0.01 for reduced FI-30-AQ. As was observed for AQ-NHS in buffer, the effect of NaBH₄ on the FI-30-AQ duplex is reversible with oxygen. Upon deliberate exposure of the reduced sample to air (i.e. oxygen) the normalized fluorescence decreased to 0.42±0.03.

In order to be able to design more complicated pseudo-electronic devices from DNA, it is necessary to not only synthesize branched structures, but also to demonstrate electron transfer through the resulting junctions. A 90 base pair Y-branched three-way DNA junction was prepared from three complementary 60 base oligonucleotides such that each arm of the junction is a 30 base pair duplex. Table 3 gives the normalized fluorescence observed for various donor-acceptor combinations for the Y-branched DNA duplexes under M-DNA conditions. The combination of one donor with two acceptors results in the greatest amount of quenching, regardless of acceptor combination, i.e., two rhodamine groups (58%) or one rhodamine and one anthraquinone (63%), and is comparable to that observed for a 54 base pair fluorescein/rhodamine labeled unbranched duplex (57% (24)). This implies that the quenching mechanism, specifically electron transfer, is in no way hindered by a branched junction in M-DNA. In contrast charge transfer through unstacked bases or through a branch or junction in B-DNA is either hindered(32-34), or does not occur(43).

Less quenching was observed for the case of a single acceptor (on average 36%), which combined with the results obtained for two acceptor molecules has two important implications. The first is that in considering the case of two acceptor molecule, there is an equal probability for electron transfer to either acceptor arm. However, the second result, namely the observed quenching for a single acceptor being less than half that for two acceptors, indicates that for the case of one acceptor molecule there is an increased probability for transfer to the acceptor-labeled arm. If it were the case that the probability of transfer to the unlabeled arm was zero, one would expect the observed quenching to again be similar to that observed for the double labeled 54 base pair unbranched duplex. However, if there is an equal probability of electron transfer to the unlabeled arm this begs the question; what is the fate of the electron once it reaches the unlabeled arm? Or is it the case that for a single acceptor molecule is there is a reduced probability for fluorescein to donate an electron as a result of quantum effects not yet considered? Regardless of the answer to these questions, the results indicate an enormous potential for the application of these systems to the design of molecular scale electronic devices.

Anthraquinone quenches fluorescein by 23% in the Y-branched duplexes, compared to rhodamine which quenches, on average, 38% (independent of which strand has the donor and acceptor chromaphore). Therefore, as for the 30 base pair duplexes, in the Y-branched DNA duplexes anthraquinone is not as efficient an acceptor as rhodamine. Nevertheless the addition of NaBH₄ again results in an increase in fluorescence emission from fluorescein, i.e., the quenching mechanism is again blocked, with the normalized emission increasing to nearly 1. For the double-labeled systems the addition of NaBH₄ results in a decrease in quenching from 63% to 38%. This provides a means to modulate the fluorescence from the Y-branched duplex, in effect mimicking the classical transistor, which consists of a source, a gate, and a drain. The source and drain electrodes are separated by a semiconducting channel, across which the potential is controlled by the gate voltage. In the Y-branched duplexes, the fluorescein-labeled arm acts as the source, and the rhodamine-labeled arm can be thought of as the drain with the anthraquinone-labeled arm acting as the gate. The state of the anthraquinone group, i.e. reduced or unreduced, provides the means of modulating the resulting signal, in this case the emission intensity from fluorescein.

TABLE 1
30- and 60-base pair sequences.

| | |
|---|---|
| ID | Sequence |

| | |
|---|---|
| FI-30 | 5′-GTG GCT AAC TAC GCA TTC CAC GAC CAA |
| | ATG-3′ |
| AQ-30/Rh-30 | 5′-CAT TTG GTC GTG GAA TGC GTA GTT AGC |
| | GAC-3′ |
| X | 5′-GCC TAG CAT GGA CTA GCG AAT TCC CGC |
| | TCT TCT CAA CTC TAG ACT CGA GGT TCC TGT |
| | CGC-3′ |
| Y | 5′-GCG TAG CCT ACG GAC TGA AGC TTA GCA |
| | GCG AGA GCG GGA ATT CGC TAG TCC ATG CTA |
| | GGC-3′ |
| Z | 5′-GCG ACA GGA ACC TCG AGT CTA GAG TTG |
| | AGA CGC TGC TAA GCT TCA GTC CGT AGG CTA |
| | CGC-3′ |

TABLE 2
Normalized fluorescence (λ_{Em} = 520 nm) for various donor-
acceptor combinations for the 30 base pair DNA duplexes;
[Zn²⁺] = 0.2 mM, pH 8.5, 20 mM Tris-HCl buffer.

| | [NaBH₄] | Normalized |
|---|---|---|
| Duplex | (mM) | Fluorescence |
| Fl-30 | 0 | 1.00 |
| Fl-30 | 2.5 | 1.00 |
| Fl-30-Rh | 0 | 0.14 ± 0.03 |
| Fl-30-Rh | 2.5 | 0.19 ± 0.03 |
| Fl-30-AQ | 0 | 0.41 ± 0.04 |
| Fl-30-AQ | 2.5 | 0.71 ± 0.01 |
| Fl-30-Aq + O₂ | 2.5 | 0.42 ± 0.03 |

TABLE 3
Normalized fluorescence (λ_{Em} = 520 nm) for various donor-
acceptor combinations for the Y-branched DNA junctions;
[Zn²⁺] = 0.2 mM, pH 8.5, 20 mM Tris-HCl buffer.

| | | | | |
|---|---|---|---|---|
| | | | [NaBH₄] | Normalized |
| X Strand | Y Strand | Z Strand | (mM) | Fluorescence |

a) Double labeled

| | | | | |
|---|---|---|---|---|
| Rhodamine | Fluorescein | | 0 | 0.61 ± 0.03 |
| Rhodamine | | Fluorescein | 0 | 0.64 ± 0.04 |
| | Fluorescein | Rhodamine | 0 | 0.62 ± 0.03 |
| | Rhodamine | Fluorescein | 0 | 0.60 ± 0.06 |
| Fluorescein | Rhodamine | | 0 | 0.65 ± 0.03 |
| Fluorescein | | Rhodamine | 0 | 0.62 ± 0.01 |
| Fluorescein | | Rhodamine | 2.5 | 0.66 ± 0.02 |
| Fluorescein | Anthra- | | 0 | 0.77 ± 0.01 |
| | quinone | | | |
| Fluorescein | Anthra- | | 2.5 | 0.92 ± 0.02 |
| | quinone | | | |

b) Triple Labeled

| | | | | |
|---|---|---|---|---|
| Rhodamine | Rhodamine | Fluorescein | 0 | 0.42 ± 0.03 |
| Rhodamine | Fluorescein | Rhodamine | 0 | 0.42 ± 0.03 |
| Fluorescein | Rhodamine | Rhodamine | 0 | 0.42 ± 0.03 |
| Fluorescein | Anthra- | Rhodamine | 0 | 0.37 ± 0.01 |
| | quinone | | | |
| Fluorescein | Anthra- | Rhodamine | 2.5 | 0.62 ± 0.02 |
| | quinone | | | |

Conclusions: Anthraquinone, covalently attached to DNA, has been shown to be an efficient quencher of the fluorescence from fluorescein in M-DNA systems. Good quenching is observed over distances of 60 base pairs, through a Y-branched junction. Therefore, as has been previously suggested(9,23,24), the M-DNA conformation offers an improved pathway for efficient conduction in DNA, a critical aspect for future development of nanometer-scale electronic devices. The emission intensity from fluorescein was modulated by chemical reduction of the anthraquinone group which was reversible by reoxidation with oxygen, providing a simple chemical switch. Applying these results to the Y-branched junctions containing 1 donor and two acceptors results in a system that optically mimics an electronic transistor. As such these systems are a critical first step in the future development of more complex nanoelectronic devices.

### REFERENCES

The following documents are incorporated herein by reference:
1. Mao, C., Sun, W., Shen, Z. and Seeman, N. C. (1999) A nanomechanical device based on the B-Z transition of DNA. Nature, 397, 144-146.
2. Yan, H., Zhang, X., Shen, Z. and Seeman, N. C. (2002) A robust DNA mechanical device controlled by hybridization topology. Nature, 415, 62-65.
3. Zhang, X., Yan, H., Shen, Z. and Seeman, N. C. (2002) Paranemic cohesion of topologically-closed DNA molecules. J. Am. Chem. Soc., 124, 12940-12941.
4. Niemeyer, C. M. and Adler, M. (2002) Nanomechanical devices based on DNA. Angew. Chem. Int. Ed., 41(20), 3779-3783.
5. Arkin, M. R., Stemp, E. D. A., Holmink, R. E., Barton, J. K., Hormann, A., Olson, E. J. C. and Barbara, P. F. (1996) Rates of DNA-mediated electron transfer between metallointercalators. Science, 273, 475-479.
6. Hall, D. B., Holmlin, R. E. and Branton, J. K. (1996) Oxidative DNA damage through long-range electron transfer. Nature, 382, 731-735.
7. Dandliker, P. J., Holmlin, R. E. and Barton, J. K. (1997) Oxidative thymine dimer repair in the DNA helix. Science, 275, 1465-1468.
8. Porath, D., Bezryadin, A., de Vries, S. and Dekker, C. (2000) Direct measurement of electrical transport through DNA molecules. Nature, 403, 635-638.
9. Rakitin, A., Aich, P., Papadopoulos, C., Kobzar, Y., Vedeneev, A. S., Lee, J. S. and Xu, J. M. (2001) Metallic conduction through engineered DNA: DNA nanoelectronic building blocks. Phys. Rev. Lett., 86(16), 3670-3673.
10. Storm, A. J., van Noort, J., de Vries, S. and Dekker, C. (2001) Insulating behavior for DNA molecules between nanoelectrodes at the 100 nm length scale. Appl. Phys. Lett., 79, 3881-3883.
11. Gomez-Navarro, C., Moreno-Herrero, F., de Pablo, P. J., Gomez-Herrero, J. and Baro, A. M. (2002) Contactless experiments on individual DNA molecules show no evidence for molecular wire behavior. Proc. Nat. Acad. Sci. (USA), 99(13), 8484-8487.
12. Braun, E., Eichen, Y., Sivan, U. and Ben-Yoseph, G. (1998) DNA-templated assembly and electrode attachment of a conducting silver wire. Nature, 391, 775-778.
13. Hurley, D. J. and Tor, Y. (2002) Donor/Acceptor interactions in systematically modified Ru^{II}—Os^{II} oligonucleotides. J. Am. Chem. Soc., 124, 13231-13241.
14. O'Neill, M. A. and Barton, J. K. (2002) 2-Aminopurine: A probe of structural dynamic and charge transfer in DNA and DNA:RNA hybrids. J. Am. Chem. Soc., 124, 13053-13066.
15. Henderson, P. T., Jones, D., Hampikian, G., Kan, Y. and Schuster, G. B. (1999) Long-distance charge transport in duplex DNA: The phonon assisted polaron-like hopping mechanism. Proc. Nat. Acad. Sci. (USA), 96, 8353-8358.
16. Jortner, J., Bixon, M., Langenbacher, T. and Michel-Beyerle, M. E. (1998) Charge transfer and transport in DNA. Proc. Nat Acad. Sci. (USA), 95, 12759-12765.
17. Bixon, M. and Jortner, J. (2001) Charge transport in DNA via thermally induced hopping. J. Am. Chem. Soc., 123, 12556-12567.
18. Bixon, M., Giese, B., Wessely, S., Langenbacher, T., Michel-Beyerle, M. E. and Jortner, J. (1999) Long-range charge hopping in DNA. Proc. Nat. Acad. Sci. (USA), 96(21), 11713-11716.
19. Meggers, E., Michel-Beyerle, M. E. and Giese, B. (1998) Sequence dependent long range hole transport in DNA. J. Am. Chem. Soc., 120, 12950-12955.
20. Giese, B., Amaudrut, J., Kohler, A.-K., Spormann, M. and Wessely, S. (2001) Direct observation of hole transfer between adenine bases and by tunnelling. Nature, 412(318-320).
21. Hess, S., Gotz, M., Davis, W. B. and Michel-Beyerle, M.-E. (2001) On the apparently anomalous distance dependence of charge-transfer rates in 9-amino-6-chloro-2-methoxyacridine-modified DNA. J. Am. Chem. Soc., 123, 10046-10055.
22. Pascaly, M., Yoo, J. and Barton, J., K. (2002) DNA mediated charge transport: Characteristics of a DNA radial localised at an artificial nucleic acid base. J. Am. Chem. Soc., 124(9083-9092).
23. Aich, P., Labiuk, S., L., Tari, L., W., Delbaere, L., J. T., Roesler, W., J., Falk, K. J., Steer, R. P. and Lee, J. S. (1999) M-DNA: A complex between divalent metal ions and DNA which behaves as a molecular Wire. J. Mol. Biol., 294, 477-485.
24. Aich, P., Skinner, R. J. S., Wettig, S. D., Steer, R. P. and Lee, J. S. (2002) Long range molecular wire behaviour in a metal complex of DNA. J. Biomol. Struct Dynamics, 20, 1-6.
25. Lee, J. S., Latimer, L., J. P. and Reid, R. S. (1993) A cooperative conformations change in duplex DNA induced by Zn²⁺ and other divalent metal ions. Biochem. Cell Bio., 71, 162-168.
26. Gasper, S. M. (1997) Intramolecular photoinduced electron transfer to anthraquinones linked to duplex DNA: The effect of gaps and traps on long-range radical cation migration. J. Am. Chem. Soc., 119, 12762-12771.
27. Ly, D., Kan, Y., Armitage, B. and Schuster, G. B. (1996) Cleavage of DNA by irradiation of substituted anthraquinones: Intercalation promotes electron transfer and efficient raction at GG steps. J. Am. Chem. Soc., 118(8747-8748).
28. Armitage, B., Yu, C., Devadoss, C. and Schuster, G., B. (1994) Cationic anthraquinone derivatives as catalytic DNA photonucleases: Mechanism for DNA damage and quinone recycling. J. Am. Chem. Soc., 116, 9487-9859.
29. Rajesh, C. S., Capitosti, G. J., Cramer, S. J. and Modarelli, D. A. (2001) Photoinduced Electron-Transfer within Free Base and Zinc Porphyrin Containing Poly(Amide) Dendrimers. J. Phys. Chem. B, 105, 10175-10188.
30. Lilley, D. M. J. (2000) Structures of helical junctions in nucleic acids. Quart. Rev. Biophys., 33, 109-159.
31. Duckett, D. R. and Lilley, D. M. J. (1990) The three-way DNA junction is a Y-shaped molecule in which there is no helix-helix stacking. EMBO Journal, 9, 1659-1664.
32. Fahlman, R. P. and Sen, D. (2002) DNA conformational switches as sensitive electronic sensors of analytes. J. Am. Chem. Soc., 124, 4610-4616.
33. Giese, B. and Wessely, S. (2000) The Influence of Mismatches on Long-Distance Charge Transport through DNA. Angew. Chem. Int. Ed., 39, 93490-3491.
34. Kelley, S. O., Holmlin, R. E., Stemp, E. D. A. and K., B. J. (1997) Photoinduced Electron Transfer in Ethidium-Modified DNA Duplexes: Dependence on Distance and Base Stacking. J. Am. Chem. Soc., 117, 9861-9870.
35. Wightman, R. M., Cockrell, J. R., Murray, R., W., Burnett, J. N. and Jones, S. B. (1976) Protonation kinetics and mechanisms for 1,8-dihydroxyanthraquinone and anthraquinone anion radicals in dimethylformamide solvent. J. Am. Chem. Soc., 98(9), 2562-2570.
36. Liu, M. D., Patterson, D. H., Jones, C. R. and Leidner, C. R. (1991) Redox and structural properties of quinone functionalized phosphatidylcholine liposomes. J. Phys. Chem., 95, 1858-1865.
37. Wettig, S. D., Li, C., Long, Y., Kraatz, H.-B. and Lee, J. S. (2002) “21st century bioanalysis” M-DNA: a self-assembling molecular wire for nanoelectronics and biosensing. Anal. Sci., Accepted.
38. Lakowicz, J. R. (1999) Principles of fluorescence spectroscopy, 2 Ed., Kluwer Academic/Plenum Publishers, New York.
39. Loufty, R. O. and Sharp, J. H. (1976) Correlation between photographic properties of dyes and their electrochemical and spectroscopic parameters. Photographic Science and Engineering, 20, 165-174.
40. Chanon, M. and Eberson, L. (1988) Photochemistry of homogeneous and heterogeneous chemical gears involving electron transfer catalysis: Chains, catalysts and sensitization. Relations to electrochemistry, synthetic applications and mechanistic basis for selectivity. In Fox, M. A., and Chanon, M. (eds.), Photoinduced Electron Transfer Part A: Conceptual Basis. Elsevier, New York, pp. 409-597.
41. Fox, M. A. (1990) Photoinduced electron transfer. Photochem. Photobiol., 52(3), 617-627.
42. Zhang, H., Zhou, Y., Zhang, M., Shen, T., Li, Y. and Zhu, D. (2002) A Comparative Study on Photo-Induced Electron Transfer from Fluorescein to Anthraquinone and Injection into Colloidal TiO₂. J. Colloid Interface Sci., 251, 443-446.
43. Fahlman, R. P., Sharma, R. D. and Sen, D. (2002) The charge conduction properties of DNA Holliday junctions depend critically on the identity of the tethered photooxidant. J. Am. Chem. Soc., 124, 12477-12485.

### FURTHER REFERENCES

All of the following documents are incorporated herein by reference:
a. Gelbart, W. M., Bruinsma, R. F., Pinkcus, P. A., and Parsegian, V. A. Physics Today 53, September 2000, 38-44 (2000).
b. Porath, D, Bezryadin, A., de Vries, S., and Dekker, C. Nature 403, 635-638 (2000).
c. Lewis, F. D., Wu, T., Zhang, Y., Letsinger, R. L., Greenfield, S. R., and Wasielewski, M. R. Science 277, 673-676 (1997).
d. Taubes, G. Science 275, 1420-1421 (1997).
e. Aich, P., Labiuk, S. L., Tarl L. W., Delbaere, L. J. T., Roesler, W. J., Faulk, K. J., Steer, R. P., and Lee, J. S. Journal of Molecular Biology 294, 477-485 (1999).
f. Lee, J. S., Latimer, L. J. P., and Reid, R. S. Biochem. Cell Biol. 71, 162-168 (1993).
g. Braun, E., Eicher, Y., Sivan, U., and Ber-Yoseph, G. Nature 391, 775-778 (1998).
h. Lines, M. E., and Glass, A. M. Principles & Applications of Ferroelectrics & Related Materials (Clarendon Press, Oxford, 1977).
i. Sponer, J., Burda, J. V., Leszczynski, J., and Hobza, P. J. Biomol. Struct. Dyn. 17, 61 (1999).
j. Seeman, N. C., and Kallenback, N. R. Ann. Rev. Biophys. Biomol. Struct. 23, 53-86 (1994).
k. Wang, J., Rivas, G., Jiang, M., and Zhang, X. Langmuir. 15, 6541-6545 (1999).
l. Aich and Lee (1999) WO 99/31115

1. An organic circuit element comprising:
a) a plurality of members, each of which comprises an oligonucleotide duplex, said plurality of members comprising:
i) at least one donor member for receiving conduction electrons from an electron donor;
ii) at least one acceptor member for communicating with an electron acceptor to provide a region of attraction for said conduction electrons; and
iii) at least one regulator member intersecting with at least one of said plurality of members to define at least one electric field regulation junction, for cooperating with an electric field regulator to regulate an electric field at the junction;
wherein the electron donor or the electron acceptor are adapted to be reversibly chemically modified to alter the conductivity of the organic circuit element under conditions that preserve the conductivity of the circuit element.

2. The organic circuit element of claim 1 wherein at least some of said members comprise a conductive metal-containing oligonucleotide duplex.

3. The organic circuit element of claim 1 wherein each of said members comprises a conductive metal-containing oligonucleotide duplex.

4. The organic circuit element of claim 1 wherein each of said at least one donor member and said at least one acceptor member comprises a conductive metal-containing oligonucleotide duplex.

5. The organic circuit element of claim 2 further comprising said electron donor in electrical communication with said donor member.

6. The organic circuit element of claim 2 further comprising said electron acceptor in electrical communication with said acceptor member.

7. The organic circuit element of claim 2 further comprising said electric field regulator in electrical communication with said regulator member.

8. The organic circuit element of claim 7 further comprising said electron donor in electrical communication with said donor member, and further comprising said electron acceptor in electrical communication with said acceptor member.

9. The organic circuit element of claim 2 wherein said donor member, said acceptor member and said regulator member intersect to define said electric field regulation junction.

10. The organic circuit element of claim 2 wherein said regulator member intersects with one of said donor member and said acceptor member to define said electric field regulation junction.

11. The organic circuit element of claim 2 wherein said plurality of members comprises a common member, and wherein said donor member, said acceptor member and said regulator member intersect said common member at first, second and third locations respectively, said third location defining said electric field regulation junction.

12. The organic circuit element of claim 2 wherein said at least one regulator member comprises a plurality of regulator members, said plurality of regulator members intersecting other respective members of said plurality of members to define said at least one electric field regulation junction.

13. The organic circuit element of claim 2 wherein said conductive metal-containing oligonucleotide duplex comprises a first nucleic acid strand and a second nucleic acid strand, said first and said second nucleic acid strands comprising respective pluralities of nitrogen-containing aromatic bases covalently linked by a backbone, said nitrogen-containing aromatic bases of said first nucleic acid strand being joined by hydrogen bonding to said nitrogen-containing aromatic bases of said second nucleic acid strand, said nitrogen-containing aromatic bases on said first and said second nucleic acid strands forming hydrogen-bonded base pairs in stacked arrangement along a length of said conductive metal-containing oligonucleotide duplex, said hydrogen-bonded base pairs comprising an interchelated metal cation coordinated to a nitrogen atom in one of said nitrogen-containing aromatic bases.

14. The organic circuit element of claim 13 wherein said interchelated metal cation comprises an interchelated divalent metal cation.

15. The organic circuit element of claim 14 wherein said divalent metal cation is selected from the group consisting of zinc, cobalt and nickel.

16. The organic circuit element of claim 13 wherein said metal cation is selected from the group consisting of the cations of Li, Be, Na, Mg, Al, K, Ca, Sc, Ti, V, Cr, Mn, Fe, Co, Ni, Cu, Zn, Ga, Ge, As, Rb, Sr, Y, Zr, Nb, Mo, Tc, Ru, Rh, Pd, Ag, Cd, In, Sn, Sb, Cs, Ba, La, Ce, Pr, Nd, Pm, Sm, Eu, Gd, Th, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U, Np and Pu.

17. The organic circuit element of claim 14 wherein said first and said second nucleic acid strands comprise deoxyribonucleic acid and said nitrogen-containing aromatic bases are selected from the group consisting of adenine, thymine, guanine and cytosine.

18. The organic circuit element of claim 14 wherein said divalent metal cations are substituted for imine protons of said nitrogen-containing aromatic bases, and said nitrogen-containing aromatic bases are selected from the group consisting of thymine and guanine.

19. The organic circuit element of claim 14 wherein at least one of said nitrogen-containing aromatic bases comprises thymine, having an N3 nitrogen atom, and said divalent metal cation is coordinated by said N3 nitrogen atom.

20. The organic circuit element of claim 14 wherein at least one of said nitrogen-containing aromatic bases comprises guanine, having an N1 nitrogen atom, and said divalent metal cation is coordinated by said N1 nitrogen atom.

21. The organic circuit element of claim 8 wherein said electron donor comprises an electrode operable to donate an electron to said donor member.

22. The organic circuit element of claim 8 wherein said electron acceptor comprises an electrode operable to accept an electron from said acceptor member.

23. The organic circuit element of claim 8 wherein said electron donor comprises an electron donor molecule capable of donating an electron to said donor member.

24. The organic circuit element of claim 23 wherein said electron donor molecule comprises a fluorescent molecule.

25. The organic circuit element of claim 24 wherein said electron donor molecule comprises fluorescein.

26. The organic circuit element of claim 8 wherein said electron acceptor comprises an electron acceptor molecule capable of accepting an electron from said acceptor member.

27. The organic circuit element of claim 26 wherein said electron acceptor molecule comprises a fluorescent molecule.

28. The organic circuit element of claim 27 wherein said electron acceptor molecule comprises rhodamine.

29. The organic circuit element of claim 8 wherein said electric field regulator comprises a regulator chromophore.

30. The organic circuit element of claim 8 wherein said electric field regulator comprises a fluorescent molecule

31. The organic circuit element of claim 8 wherein said electric field regulator comprises a fluorescein.

32. The organic circuit element of claim 8 wherein said electric field regulator comprises a rhodamine.

33. The organic circuit element of claim 29 wherein said regulator chromophore absorbs radiation within a range of wavelengths.

34. The organic circuit element of claim 8 wherein said electron acceptor comprises a chromophore operable to emit radiation within a range of wavelengths in response to accepting an electron from said acceptor member.

35. The organic circuit element of claim 8 wherein said electric field regulator comprises an electrode.

36. The organic circuit element of claim 8 wherein said electric field regulator comprises a plurality of states, each state of said plurality of states being selectable to produce a respective electrostatic potential at said electric field regulation junction.

37. The organic circuit element of claim 36 wherein said states are selectable in response to an applied external potential.

38. A system comprising the organic circuit element of claim 8 and further comprising a conductive medium for supplying conduction electrons to said electron donor and for receiving conduction electrons from said electron acceptor.

39. The system of claim 38 wherein said conductive medium is operable to donate electrons to said electron donor, and is operable to accept electrons from said electron acceptor to provide a closed circuitway for electrons to flow from said electron donor, through said donor member, through said electric field regulation junction, through said acceptor member, through said electron acceptor, and back to said electron donor.

40. The system of claim 39 wherein said conductive medium comprises an aqueous solution.

41. The system of claim 39 wherein said conductive medium comprises a conductive wire.

42. A method of making an organic circuit element, the method comprising annealing and treating a plurality of oligonucleotides to form a plurality of members, each member of said plurality of members comprising a pair of said oligonucleotides aligned to form a duplex portion, said plurality of members comprising:
a) at least one donor member for receiving conduction electrons from an electron donor;
b) at least one acceptor member for communicating with an electron acceptor to provide a region of attraction for said conduction electrons; and
c) at least one regulator member intersecting with at least one of said plurality of members to define at least one electric field regulation junction, for cooperating with an electric field regulator to regulate an electric field at the junction.

43.-81. (canceled)

82. A method of regulating an electronic signal between first and second locations in a conductive nucleic acid material, the method comprising varying an electrostatic potential at a third location in the nucleic acid material interposed between the first and second locations.

83. The method of claim 82 wherein varying comprises selecting one of a plurality of states of an electric field regulator in communication with the third location, each of the states corresponding to a respective electrostatic potential at the third location.

84.-94. (canceled)

95. The method of claim 82 wherein the first location comprises a location in a conductive nucleic acid electron donor member, the second location comprises a location in a conductive nucleic acid electron acceptor member, and the third location comprises at least one electric field regulation junction in electrical communication with the donor member and the acceptor member, and wherein varying comprises varying the electrostatic potential at the at least one electric field regulation junction.

96. The method of claim 95 wherein the at least one electric field regulation junction is in electrical communication with a conductive nucleic acid electric field regulator member, and wherein varying comprises selecting one of a plurality of states of an electric field regulator in electrical communication with the regulator member, each of the states corresponding to a respective electrostatic potential at the at least one electric field regulation junction.

97.-109. (canceled)

110. The method of claim 82 wherein the conductive nucleic acid material comprises a plurality of members, each of which comprises a conductive metal-containing oligonucleotide duplex, said plurality of members comprising at least one donor member for receiving conduction electrons from an electron donor, at least one acceptor member for communicating with an electron acceptor to provide a region of attraction for said conduction electrons, and at least one regulator member intersecting with at least one of said plurality of members to define at least one electric field regulation junction, for cooperating with an electric field regulator to regulate an electric field at the junction; and wherein varying comprises selecting one of a plurality of states of the electric field regulator, each of the states corresponding to a respective electrostatic potential at the electric field regulation junction.

111. The method of claim 82 wherein the conductive nucleic acid material comprises a conductive metal-containing nucleic acid duplex, said conductive metal-containing nucleic acid duplex comprising a regulator member in electrical communication with an electric field regulator, a donor member in electrical communication with an electron donor, and an acceptor member in electrical communication with an electron acceptor, and wherein varying comprises changing a state of said electric field regulator to vary an electrostatic potential at an electric field regulation junction joining said regulator member, said donor member, and said acceptor member, to regulate the signal.

112. The method of claim 111 wherein said conductive metal-containing nucleic acid duplex comprises a nucleic acid duplex comprising a first nucleic acid strand and a second nucleic acid strand, said first and said second nucleic acid strands comprising respective pluralities of nitrogen-containing aromatic bases covalently linked by a backbone, said nitrogen-containing aromatic bases of said first nucleic acid strand being joined by hydrogen bonding to said nitrogen-containing aromatic bases of said second nucleic acid strand, said nitrogen-containing aromatic bases on said first and said second nucleic acid strands forming hydrogen-bonded base pairs in stacked arrangement along a length of said nucleic acid duplex.

113. The method of claim 112 further comprising producing said conductive metal-containing nucleic acid duplex.

114. The method of claim 113 wherein producing comprises subjecting said nucleic acid duplex to a basic solution in the presence of a metal cation under conditions effective to form said conductive metal-containing nucleic acid duplex, wherein said hydrogen-bonded base pairs of said conductive metal-containing nucleic acid duplex comprise an interchelated metal cation coordinated to a nitrogen atom in one of said nitrogen-containing aromatic bases.

115.-147. (canceled)

148. An apparatus for regulating an electronic signal between first and second locations in a conductive nucleic acid material, the apparatus comprising:
a) the conductive nucleic acid material having the first and second locations; and
b) means for varying an electrostatic potential at a third location in the nucleic acid material interposed between the first and second locations.

149. The apparatus of claim 148 wherein said means for varying comprises means for selecting one of a plurality of states of an electric field regulator in communication with the third location, each of the states corresponding to a respective electrostatic potential at the third location.

150. The apparatus of claim 149 wherein said means for selecting comprises means for irradiating the electric field regulator.

151. The apparatus of claim 149 wherein said means for selecting comprises means for applying an external potential to the electric field regulator.

152. The apparatus of claim 151 wherein said electric field regulator comprises an electrode, and wherein said means for applying comprises means for depositing at least one electron onto said electrode to apply a negative electrostatic potential to the third location.

153. (canceled)

154. (canceled)

155. The apparatus of claim 148 wherein the first location comprises a location in a conductive nucleic acid electron donor member, the second location comprises a location in a conductive nucleic acid electron acceptor member, and the third location comprises at least one electric field regulation junction in electrical communication with the donor member and the acceptor member, and wherein said means for varying comprises means for varying the electrostatic potential at the at least one electric field regulation junction.

156. The apparatus of claim 155 wherein the at least one electric field regulation junction is in electrical communication with a conductive nucleic acid electric field regulator member, and wherein said means for varying comprises means for selecting one of a plurality of states of an electric field regulator in electrical communication with the regulator member, each of the states corresponding to a respective electrostatic potential at the at least one electric field regulation junction.

157. The apparatus of claim 156 wherein said means for selecting comprises means for irradiating the electric field regulator.

158. The apparatus of claim 156 wherein said means for selecting comprises means for applying an external potential to the electric field regulator.

159. The apparatus of claim 158 wherein said electric field regulator comprises an electrode, and wherein said means for applying comprises means for depositing at least one electron onto said electrode to apply a negative electrostatic potential to said electric field regulation junction, said negative electrostatic potential decreasing the ability of an electron to travel from said donor member to said acceptor member.

160. The apparatus of claim 158 wherein said electric field regulator comprises an electrode, and wherein said means for applying comprises means for removing at least one electron from said electrode to apply a positive electrostatic potential to said electric field regulation junction, said positive electrostatic potential increasing the ability of an electron to travel from said donor member to said acceptor member.

161. The apparatus of claim 155 further comprising means for producing the electronic signal.

162. An apparatus for regulating an electronic signal between first and second locations in a conductive nucleic acid material, the apparatus comprising an electric field regulator operable to vary an electrostatic potential at a third location in the nucleic acid material interposed between the first and second locations.

163.-169. (canceled)

170. The apparatus of claim 162 wherein the first location comprises a location in a conductive nucleic acid electron donor member, the second location comprises a location in a conductive nucleic acid electron acceptor member, and the third location comprises at least one electric field regulation junction in electrical communication with the donor member, the acceptor member, and said electric field regulator.

171. (canceled)

172. A method of regulating an electronic signal in a conductive nucleic acid material, the method comprising varying a degree of electric field regulation at an electric field regulation junction at which a regulator member intersects at least one of a plurality of members, each of said regulator member and said plurality of members comprising an oligonucleotide duplex and at least some of said regulator member and said plurality of members comprising a conductive metal-containing oligonucleotide duplex, said plurality of members comprising at least one donor member for receiving conduction electrons from an electron donor, and at least one acceptor member for communicating with an electron acceptor to provide a region of attraction for said conduction electrons.

173.-176. (canceled)

177. A method of storing data, the method comprising selecting one of at least two states of an electric field regulator of a nucleic acid circuit element, each of said at least two states corresponding to a respective degree of electric field regulation at an electric field regulation junction in the circuit element, each said degree of electric field regulation corresponding to a respective data value.

178. (canceled)

179. (canceled)

180. The method of claim 177 wherein said nucleic acid circuit element comprises a plurality of members, at least some of which comprise a conductive metal-containing oligonucleotide duplex, said plurality of members comprising at least one donor member for receiving conduction electrons from an electron donor, at least one acceptor member for communicating with an electron acceptor to provide a region of attraction for said conduction electrons, and at least one regulator member intersecting with at least one of said plurality of members to define said electric field regulation junction, said regulator member being in communication with said electric field regulator, and wherein selecting comprises causing said electric field regulation junction to apply said degree of electric field regulation to the electric field regulation junction, to represent said data value.

181. The method of claim 180 wherein causing comprises selecting one of a plurality of states of said electric field regulator, each of said states corresponding to a respective electrostatic potential at said electric field regulation junction.

182. An organic data storage medium comprising an electric field regulator having at least two selectable states, each of the states corresponding to a respective degree of electric field regulation at an electric field regulation junction of a nucleic acid circuit element, each said degree of electric field regulation corresponding to a respective data value.

183. The organic data storage medium of claim 182 further comprising said nucleic acid circuit element, said nucleic acid circuit element comprising a plurality of members, at least some of which comprise a conductive metal-containing oligonucleotide duplex, said plurality of members comprising:
a) at least one donor member for receiving conduction electrons from an electron donor;
b) at least one acceptor member for communicating with an electron acceptor to provide a region of attraction for said conduction electrons; and
c) at least one regulator member intersecting with at least one of said plurality of members to define said electric field regulation junction, for cooperating with said electric field regulator to apply said degree of electric field regulation to said junction, to represent said data value.

184.-186. (canceled)

187. An apparatus for storing data, the apparatus comprising:
a) a conductive nucleic acid circuit element comprising an electric field regulation junction; and
b) means for varying a degree of electric field regulation at said electric field regulation junction in said circuit element, each said degree of electric field regulation corresponding to a respective data value.

188. (canceled)

189. An electrical conductor comprising an electron source electrically coupled to a conductive metal-containing nucleic acid duplex, the conductive metal-containing nucleic acid duplex comprising a first strand of nucleic acid and a second strand of nucleic acid, the first and the second nucleic acid strands comprising a plurality of nitrogen-containing aromatic bases covalently linked by a backbone, the nitrogen-containing aromatic bases of the first nucleic acid strand being joined by hydrogen bonding to the nitrogen-containing aromatic bases of the second nucleic acid strand, the nitrogen-containing aromatic bases on the first and the second nucleic acid strands forming hydrogen-bonded base pairs in stacked arrangement along the length of the conductive metal-containing nucleic acid duplex, the hydrogen-bonded base pairs comprising an interchelated divalent metal cation coordinated to a nitrogen atom in one of the aromatic nitrogen-containing aromatic bases, to form the electrical conductor, further comprising an electron sink electrically coupled to the conductive metal-containing nucleic acid duplex, wherein the electron source is a molecule capable of donating an electron to the conductive metal-containing nucleic acid duplex, and the electron sink is an electron acceptor molecule capable of accepting an electron from the conductive metal-containing nucleic acid duplex, and wherein the electron donor or the electron acceptor are adapted to be reversibly chemically modified to alter the conductivity of the organic circuit element under conditions that preserve the conductivity of the circuit element.


SEQUENCE LISTING (5 sequences)

SEQ ID NO: 1 - DNA, 30 bp, Artificial sequence
  synthetic oligonucleotide, ID = Fl-30
gtggctaactacgcattccacgaccaaatg

SEQ ID NO: 2 - DNA, 30 bp, Artificial sequence
  synthetic oligonucleotide,  ID = AQ-30/Rh-30
catttggtcgtggaatgcgtagttagccac

SEQ ID NO: 3 - DNA, 60 bp, Artificial sequence
  synthetic oligonucleotide,  ID = X
gcctagcatggactagcgaattcccgctcttctcaactctagactcgaggttcctgtcgc

SEQ ID NO: 4 - DNA, 60 bp, Artificial sequence
  synthetic oligonucleotide,  ID = Y
gcgtagcctacggactgaagcttagcagcgagagcgggaattcgctagtccatgctaggc

SEQ ID NO: 5 - DNA, 60 bp, Artificial sequence
  synthetic oligonucleotidem  ID = Z
gcgacaggaacctcgagtctagagttgagacgctgctaagcttcagtccgtaggctacgc